(19) United States
(12) Patent Application Publication
(10) Pub. No.: US 2026/0062813 A1
(43) Pub. Date: Mar. 5, 2026

(54) THIOSEMICARBAZONE CORROSION INHIBITORS

(71) Applicants: Iowa State University Research Foundation, Inc., Ames, IA (US); University of Washington, Seattle, WA (US)

(72) Inventors: George A. Kraus, Ames, IA (US); Jean-Philippe Tessonnier, Ames, IA (US); Demetrius Finley, Detroit, MI (US); Krishna Sai Koushik Achanta, Ames, IA (US); Jim Pfaendtner, Raleigh, NC (US); Nadia Intan, Richland, WA (US)

(21) Appl. No.: 19/316,276

(22) Filed: Sep. 2, 2025

Related U.S. Application Data
(60) Provisional application No. 63/690,354, filed on Sep. 4, 2024.

(51) Int. Cl. C23F 11/04; C07D 239/42; C07D 471/04

(52) U.S. Cl. CPC C23F 11/04; C07D 239/42; C07D 471/04

(57) ABSTRACT

A compound for corrosion inhibition of a metal is a thiosemicarbazone. A method of inhibition corrosion includes contacting a metal with the thiosemicarbazone and/or a reaction product thereof.

### CROSS-REFERENCE TO RELATED APPLICATION

This application claims the benefit of priority to U.S. Provisional Patent Application Ser. No. 63/690,354 filed Sep. 4, 2024, the disclosure of which is incorporated herein in its entirety by reference.

### STATEMENT OF GOVERNMENT SUPPORT

This invention was made with Government support under DE-EE0008492 awarded by the DOE. The Government has certain rights in the invention.

### BACKGROUND

Metal corrosion has an estimated global cost of $2.5 trillion per year. It impacts every industrial sector, with the energy sector being disproportionally affected due to the prevalence of mild steel for the construction of industrial plants, storage tanks, and pipelines. A plethora of both inorganic and organic corrosion inhibitors have been developed over the past decades, but there are growing concerns about their toxicity and impact on the environment and human health.

Mild steel stands as one of the most prevalent construction materials, finding applications in various sectors, including construction, energy, manufacturing, and transportation. Its utilization is particularly pronounced in the energy sector, where it serves in the fabrication of pipelines, containers, and related infrastructure, primarily due to its cost-effectiveness and robust mechanical properties. However, its corrosion remains a major challenge, especially in the case of pipelines. The transportation and storage of crude oil, which is filled with copious minerals, often results in the accumulation of deposits along the pipelines and containers, causing their blockage. To suppress such deposits, the oil industry utilizes an array of processes that include acid pickling and descaling, where potent acids such as hydrochloric acid (HCl) and sulfuric acid (H₂SO₄) are employed to dissolve the inorganic deposits. Although these processes are effective in cleaning the pipelines, they also affect mild steel, which is prone to corrosion in the presence of strong acids.

Corrosion inhibitors play a vital role in protecting and preventing the deterioration of mild steel. Traditionally, pipelines are protected by the application of inorganic coatings that contain compounds such as chromates, molybdates, and the like. However, these compounds are carcinogenic and detrimental to the environment. Hence, new strategies for the synthesis of eco-friendly inhibitors need to be outlined and explored.

### SUMMARY OF THE INVENTION

Various aspects of the present disclosure provide a compound for corrosion inhibition of a metal. The compound can be an indole-3-thiosemicarbazone. In various aspects, the compound has the structure:

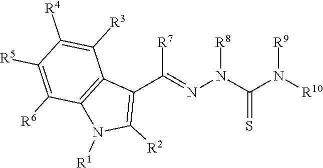

The variables R¹-R³ and R⁵-R⁸ are independently chosen from are independently chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, thiazol-2-yl, pyrimidin-2-yl, benzo[d]thiazol-2-yl, —Cl, —Br, —F, and —I. The variable R⁴ is chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, thiazol-2-yl, pyrimidin-2-yl, benzo[d]thiazol-2-yl, —Cl, —Br, —F, —I, and —C═N—NH—C(═S)—NH₂. The variables R⁹ and R¹⁰ are independently chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, 2-thiazol-2-yl, pyrimidin-2-yl, benzo[d]thiazol-2-yl, —Cl, —Br, —F, and —I, or wherein R⁹ and R¹⁰ together with the nitrogen atom to which they are attached form a morpholine ring or a thiomorpholine ring. At each occurrence, R¹¹ is independently chosen from —O—, —S—, and —NH—.

Various aspects of the present disclosure provide a compound for corrosion inhibition of a metal. The compound has the following chemical structure:

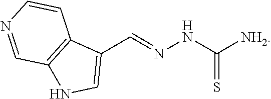 [SMILES: NC(=S)N/N=C/c1c[nH]c2cnccc12]

Various aspects of the present disclosure provide a compound for corrosion inhibition of a metal. The compound has the following chemical structure:

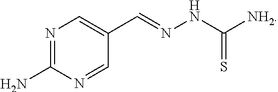 [SMILES: NC(=S)N/N=C/c1cnc(N)nc1]

Various aspects of the present disclosure provide a corrosion inhibition composition. The corrosion inhibition composition includes the corrosion inhibition compound for corrosion inhibition of a metal of the present disclosure. The corrosion inhibition composition inhibits corrosion of a metal contacted therewith.

Various aspects of the present disclosure provide a method of inhibiting corrosion. The method includes contacting the metal with the corrosion inhibition compound for corrosion inhibition of a metal of the present disclosure and/or a reaction product thereof.

Various aspects of the present disclosure provide a metal having a decreased rate of corrosion. The metal includes a coating and/or absorbed layer on an exterior surface of the metal comprising the corrosion inhibition compound for corrosion inhibition of a metal of the present disclosure or a reaction product thereof.

Various aspects of the present disclosure provide a metal having a decreased rate of corrosion. The metal includes a coating and/or absorbed layer on an exterior surface of the metal comprising a reaction product of the corrosion inhibition compound for corrosion inhibition of a metal of the present disclosure and an acidic solution, or the exterior surface of the metal, or a combination thereof.

Various aspects of the present disclosure provide a method of forming the corrosion inhibition compound for corrosion inhibition of a metal of the present disclosure. The method includes reacting a substituted or unsubstituted indole-3-carboxaldehyde with a substituted or unsubstituted thiosemicarbazide to form the corrosion inhibition compound.

Various aspects of the present disclosure have advantages over other corrosion inhibitors and methods of using and making the same. For example, in various aspects, the corrosion inhibition compound of the present disclosure can be formed from one or more sustainably derived materials, and as such can be more sustainably derived than other corrosion inhibition compounds. In various aspects, the corrosion inhibition compound of the present disclosure and/or a reaction product thereof can provide better corrosion inhibition of a metal than other corrosion inhibition compounds. In various aspects, the corrosion inhibition compound of the present disclosure and/or a reaction product thereof can provide high corrosion inhibition activity, such as corrosion inhibition efficiencies of 97.0-98.9% or higher for mild steel. In various aspects, the corrosion inhibition compound of the present disclosure or a reaction product thereof can chemisorb to the contacted metal surface, allowing treatment with the corrosion inhibition compound and/or a reaction product thereof to provide corrosion inhibition that continues to occur after removal of a solution including the corrosion inhibition compound and/or a reaction product thereof from the metal.

### BRIEF DESCRIPTION OF THE FIGURES

The drawings illustrate generally, by way of example, but not by way of limitation, various aspects of the present invention.

FIG. 1A illustrates a synthetic pathway to the thiosemicarbazones, in accordance with various aspects of the present disclosure.

FIG. 1B illustrates molecular structures of indole thiosemicarbazone (IT) corrosion inhibitors, in accordance with various aspects of the present disclosure.

FIG. 2A illustrates a Nyquist plot for mild steel in 0.1 M H₂SO₄ in the absence and presence of 1 mM of IT corrosion inhibitors, in accordance with various aspects of the present disclosure.

FIG. 2B illustrates an equivalent Randell's circuit for mild steel in 0.1 M H₂SO₄ in the absence and presence of 1 mM of IT corrosion inhibitors, in accordance with various aspects of the present disclosure.

FIG. 2C illustrates a Bode impedance magnitude plot for mild steel in 0.1 M H₂SO₄ in the absence and presence of 1 mM of IT corrosion inhibitors, in accordance with various aspects of the present disclosure.

FIG. 2D illustrates a Bode phase angle plot for mild steel in 0.1 M H₂SO₄ in the absence and presence of 1 mM of IT corrosion inhibitors, in accordance with various aspects of the present disclosure.

FIG. 3 illustrates polarization curves of mild steel coupons in 0.1 M H₂SO₄ in the presence and absence of various corrosion inhibitors, in accordance with various aspects of the present disclosure.

FIG. 4A illustrates Langmuir adsorption isotherms for inhibitor IT-1, in accordance with various aspects of the present disclosure.

FIG. 4B illustrates Langmuir adsorption isotherms for inhibitor IT-2, in accordance with various aspects of the present disclosure.

FIG. 4C illustrates Langmuir adsorption isotherms for inhibitor IT-3, in accordance with various aspects of the present disclosure.

FIG. 4D illustrates Langmuir adsorption isotherms for inhibitor IT-4, in accordance with various aspects of the present disclosure.

FIG. 5A illustrates an Arrhenius plot (log Ø vs. 1000/T) generated from the gravimetric analysis of mild steel coupons in the presence of inhibitors IT-1, IT-2, IT-3, and IT-4, in accordance with various aspects of the present disclosure.

FIG. 5B illustrates a transition state plot (log Ø/T vs. 1000/T) generated from the gravimetric analysis of mild steel coupons in the presence of inhibitors IT-1, IT-2, IT-3, and IT-4, in accordance with various aspects of the present disclosure.

FIG. 6A illustrates SEM images of mild steel coupons before and after corrosion for 1 h in 0.1 M H₂SO₄ in the absence of inhibitor, in accordance with various aspects of the present disclosure.

FIG. 6B illustrates SEM images of mild steel coupons before and after corrosion for 1 h in 0.1 M H₂SO₄ in the presence of IT-1, in accordance with various aspects of the present disclosure.

FIG. 6C illustrates SEM images of mild steel coupons before and after corrosion for 1 h in 0.1 M H₂SO₄ in the presence of IT-2, in accordance with various aspects of the present disclosure.

FIG. 6D illustrates SEM images of mild steel coupons before and after corrosion for 1 h in 0.1 M H₂SO₄ in the presence of IT-3, in accordance with various aspects of the present disclosure.

FIG. 6E illustrates SEM images of mild steel coupons before and after corrosion for 1 h in 0.1 M H₂SO₄ in the presence of IT-4, in accordance with various aspects of the present disclosure.

FIG. 7A illustrates a SEM image of a mild steel coupon corroded in the presence of IT-3, in accordance with various aspects of the present disclosure.

FIG. 7B illustrates a selected-area EDS spectrum of a mild steel coupon corroded in the presence of IT-3, in accordance with various aspects of the present disclosure.

FIG. 7C illustrates a SEM image of a mild steel coupon corroded in the presence of IT-4, in accordance with various aspects of the present disclosure.

FIG. 7D illustrates a selected-area EDS spectrum of a mild steel coupon corroded in the presence of IT-4, in accordance with various aspects of the present disclosure.

FIG. 8 illustrates electrochemical impedance spectroscopy data for compounds IT-1, 1a, IT-2, 1b, and IT-5-IT-8, in accordance with various aspects of the present disclosure.

FIG. 9 illustrates corrosion current data for compounds 1a, 1b, and IT-5-IT-8, in accordance with various aspects of the present disclosure.

FIG. 10A illustrates Langmuir isotherms for compound 1a, in accordance with various aspects of the present disclosure.

FIG. 10B illustrates Langmuir isotherms for compound 1b, in accordance with various aspects of the present disclosure.

FIG. 10C illustrates Langmuir isotherms for compound IT-5, in accordance with various aspects of the present disclosure.

FIG. 10D illustrates Langmuir isotherms for compound IT-6, in accordance with various aspects of the present disclosure.

FIG. 10E illustrates Langmuir isotherms for compound IT-7, in accordance with various aspects of the present disclosure.

FIG. 10F illustrates Langmuir isotherms for compound IT-8, in accordance with various aspects of the present disclosure.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in a specific order as recited herein. Alternatively, in any aspect(s) disclosed herein, specific acts may be carried out in any order without departing from the principles of the invention, except when a temporal or operational sequence is explicitly recited. Furthermore, specified acts can be carried out concurrently unless explicit claim language recites that they be carried out separately or the plain meaning of the claims would require it. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. A hydrocarbylene group is a diradical hydrocarbon, e.g., a hydrocarbon that is bonded at two locations.

### Corrosion Inhibition Compound.

Various aspects of the present disclosure provide a compound for corrosion inhibition of a metal that is a semicarbazone. The metal can be any suitable metal that can experience corrosion in the absence of the corrosion inhibition compound or reaction product thereof. For example, the metal can include aluminum, copper, iron, zinc, manganese, nickel, chromium, molybdenum, boron, titanium, vanadium, tungsten, cobalt, niobium, an alloy thereof, or a combination thereof. The metal can include aluminum, copper, iron, zinc, an alloy thereof, or a combination thereof. The metal can be a steel, such as a carbon steel, an alloy steel, or a combination thereof. The metal can include iron and also include manganese, nickel, chromium, molybdenum, boron, titanium, vanadium, tungsten, cobalt, niobium, or a combination thereof.

The compound for corrosion inhibition of the metal can be a thiosemicarbazone of an indole-3-carboxaldehyde, wherein the thiosemicarbazone and the indole-3-carboxaldehyde are independently substituted or unsubstituted.

The compound for corrosion inhibition of the metal can have the structure

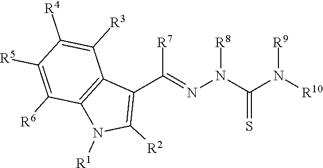

The variables R¹-R³ and R⁵-R⁸ can be independently chosen from are independently chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, thiazol-2-yl, pyrimidin-2-yl, benzo[d]thiazol-2-yl, —Cl, —Br, —F, and —I. The variable R⁴ can be chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, thiazol-2-yl, pyrimidin-2-yl, benzo[d]thiazol-2-yl, —Cl, —Br, —F, —I, and —C═N—NH—C(═S)—NH₂. The variables R⁹ and R¹⁰ can be independently chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, 2-thiazol-2-yl, pyrimidin-2-yl, benzo[d]thiazol-2-yl, —Cl, —Br, —F, and —I, or R⁹ and R¹⁰ together with the nitrogen atom to which they are attached can form a morpholine ring or a thiomorpholine ring. At each occurrence, R¹¹ can be independently chosen from —O—, —S—, and —NH—.

The variables R¹-R¹⁰ can be independently chosen from —H, —OH, —Br, (C₁-C₂₀)alkyl, —O—(C₁-C₂₀)alkyl, (C₆-C₂₀)aryl, —O—(C₆-C₂₀)aryl, (C₁-C₂₀)heteroaryl, and —O—(C₁-C₂₀)heteroaryl. The variables R¹-R¹⁰ can be independently chosen from —H, —OH, Br, —O—(C₁-C₅)alkyl, and (C₁-C₂₀)heteroaryl.

The variables R²-R³ and R⁶-R¹⁰ can be —H. The variable R⁴ and R⁵ can be independently chosen from H, —C═N—NH—C(═S)—NH₂, —OCH₃, and —Br. The variables R⁹ and R¹⁰ can be —H, or R⁹ and R¹⁰ can together form a morpholine ring. The variable R¹ can be chosen from —H and (C₁-C₂₀)heteroaryl, such as from —H, pyrimidin-2-yl, and benzo[d]thiazol-2-yl.

The variables R², R³, R⁵, R⁶, R⁷, R⁸, R⁹, and R¹⁰ can be —H. The variable R⁴ can be chosen from —H, —C═N—NH—C(═S)—NH₂, and —OCH₃. The variable R¹ can be chosen from —H, pyrimidin-2-yl, and benzo[d]thiazol-2-yl. In various aspects, R², R³, R⁵, R⁶, R⁷, R⁸, R⁹, and R¹⁰ can be —H, R¹ can be chosen from —H, pyrimidin-2-yl, and benzo[d]thiazol-2-yl, and R⁴ can be chosen from —H and —OCH₃.

The corrosion inhibition compound can have the structure:

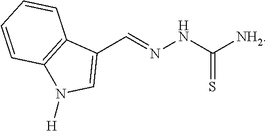 [SMILES: NC(=S)N/N=C/c1c[nH]c2ccccc12]

The corrosion inhibition compound can have the structure:

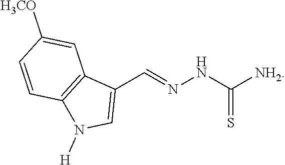 [SMILES: COc1ccc2[nH]cc(/C=N/NC(N)=S)c2c1]

The corrosion inhibition compound can have the structure:

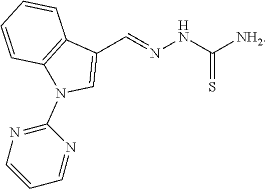 [SMILES: NC(=S)N/N=C/c1cn(-c2ncccn2)c2ccccc12]

The corrosion inhibition compound can have the structure:

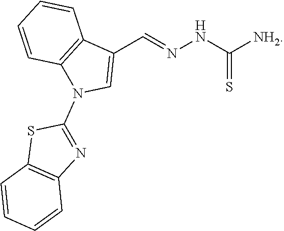 [SMILES: NC(=S)N/N=C/c1cn(-c2nc3ccccc3s2)c2ccccc12]

The corrosion inhibition compound can have the structure:

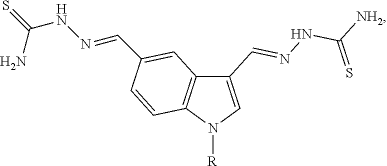

wherein R can be —H.

The corrosion inhibition compound can have the structure:

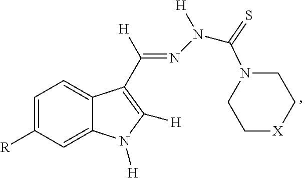

wherein R can be —H and X can be O.

In various aspects, R¹, R³, and R⁶-R¹⁰ can be —H, R² can be —H, R⁴ can be —H, and R⁵ can be —OCH₃.

In various aspects, R¹, R³, and R⁶-R¹⁰ can be —H, R² can be —H, R⁴ can be —Br, and R⁵ can be —H.

In various aspects, R¹, R³, and R⁶-R¹⁰ can be —H, R² can be —H, R⁴ can be —OH, and R⁵ can be —H.

In various aspects, R¹, R³, and R⁶-R¹⁰ can be —H, R² can be —CH₃, R⁴ can be —H, and R⁵ can be —H.

In various aspects, the compound for corrosion inhibition of a metal has the following structure:

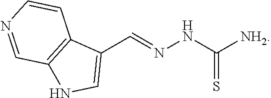 [SMILES: NC(=S)N/N=C/c1c[nH]c2cnccc12]

In various aspects, the compound for corrosion inhibition of a metal has the following structure:

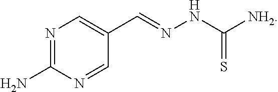 [SMILES: NC(=S)N/N=C/c1cnc(N)nc1]

The compound or a reaction product thereof can have any suitable corrosion inhibition effect on the metal. For example, the compound can have an inhibition efficiency (% η_{R}) when contacted with the metal in an aqueous solution including a concentration of 1 mM of the compound and including 0.1 M H₂SO₄ of 60 to 100, or 95 to 99.9, or 97.5 to 99.5, or less than or equal to 100 and greater than or equal to 60 and less than, equal to, or greater than 62, 64, 66, 68, 70, 72, 74, 76, 78, 80, 82, 84, 86, 88, 90, 91, 92, 93, 94, 95, 95.5, 96, 96.5, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, or 99.9.

The compound can be a corrosion inhibitor of the metal, a reaction product of the compound can be a corrosion inhibitor of the metal, or a combination thereof. In various aspects, a reaction product of the compound and (a) an acid, or (b) the metal, or (c) a combination thereof, is a corrosion inhibitor of the metal.

In various aspects, a reaction product of the compound and the metal in the presence of an acid is a corrosion inhibitor of the metal. The metal contacted with the compound under acidic conditions can exhibit inhibited corrosion. In various aspects, the metal removed from the acidic conditions can continue to exhibit inhibited corrosion.

### Corrosion Inhibition Composition.

In various aspects the present disclosure provides a corrosion inhibition composition that includes the corrosion inhibition compound of the present disclosure or a reaction product thereof, wherein the corrosion inhibition composition inhibits corrosion of a metal connected therewith. The composition can be any suitable composition that includes the corrosion inhibition composition or a reaction product thereof. The composition can be a paint, a coating, a spray, a lubricant composition (e.g., that provides increased lubricant to a material such as a metal treated therewith, a metal wear-protection composition (e.g., that provides wear inhibition of metal treated therewith), or a combination thereof.

The corrosion inhibition composition can have any suitable concentration of the corrosion inhibition compound and/or a reaction product thereof, such as 0.00001 wt % to 100 wt %, or less than or equal to 100 wt % and greater than or equal to 0.00001 wt % and less than, equal to, or greater than 0.0001 wt %, 0.001, 0.01, 0.1, 0.5, 1, 1.5, 2, 3, 4, 5, 6, 8, 10, 15, 20, 25, 30, 35, 40, 45, 50, 55, 60, 65, 70, 75, 80, 85, 90, 95, 96, 97, 98, 99, 99.5, 99.9, 99.99, or 99.999 wt %. The concentration of the compound can be 0.1 mM to 10 mM, or 0.5 mM to 1 mM, or less than or equal to 10 mM and greater than or equal to 0.1 mM and less than, equal to, or greater than 0.2 mM, 0.3, 0.4, 0.5, 0.6, 0.7, 0.8, 0.9, 1, 1.1, 1.2, 1.3, 1.4, 1.5, 1.6, 1.8, 2, 2.5, 3, 3.5, 4, 4.5, 5, 5.5, 6, 6.5, 7, 7.5, 8, 8.5, 9, or 9.5 mM.

The corrosion inhibition composition can include any suitable additional one or more components in addition to the corrosion inhibition compound and/or a reaction product thereof. For example, the composition can include water, an organic solvent, acid, base, buffer, one or more additional corrosion inhibiting compounds or additives, or a combination thereof. The composition can be an aqueous composition including an acid, such as hydrochloric acid, sulfuric acid, phosphoric acid, or a combination thereof, having a concentration of 0.001 M to 5 M, or less than or equal to 5 M and greater than or equal to 0.001 M and less than, equal to, or greater than 0.01 M, 0.02, 0.04, 0.05 0.06, 0.08, 0.1, 0.2, 0.3, 0.4, 0.5, 0.6, 0.7, 0.8, 0.9, 1, 1.2, 1.4, 1.6, 1.8, 2, 2.5, 3, 3.5, 4, or 4.5 M. The composition can have a pH of 0 to 10, or 0 to 6.5, or 0 to 2, or less than or equal to 10 and greater than or equal to 0 and less than, equal to, or greater than 0.2, 0.4, 0.6, 0.8, 1, 1.2, 1.4, 1.6, 1.8, 2, 2.5, 3, 3.5, 4, 4.5, 5, 5.5, 6, 6.5, 7, 7.5, 8, 8.5, 9, or 9.5.

The corrosion inhibition composition including the corrosion inhibition compound can have any suitable mechanism of corrosion inhibition. For example, the composition can provide inhibited corrosion of the metal during contacting of the composition with the metal or during contacting of the corrosion inhibition compound and/or a reaction product thereof with the metal. The composition can provide inhibited corrosion of the metal after contacting the composition with the metal or after contacting the corrosion inhibition compound and/or a reaction product thereof with the metal. For example, after removing the metal from contact with a composition including the compound or a reaction product thereof, the metal can still include a residual coating and/or adsorbed layer of the compound and/or a reaction product thereof on the contacted surface of the metal that inhibits corrosion of the metal.

### Method of Inhibiting Corrosion.

Various aspects of the present disclosure provide a method of inhibition corrosion of a metal. The method can include contacting the metal with the corrosion inhibition composition of the present disclosure, or contacting the metal with the corrosion inhibition compound of the present disclosure and/or a reaction product thereof. The metal can be any suitable metal that can experience corrosion in the absence of the corrosion inhibition compound or reaction product thereof. For example, the metal can include aluminum, copper, iron, zinc, manganese, nickel, chromium, molybdenum, boron, titanium, vanadium, tungsten, cobalt, niobium, an alloy thereof, or a combination thereof. The metal can include aluminum, copper, iron, zinc, an alloy thereof, or a combination thereof. The metal can be a steel, such as a carbon steel, an alloy steel, or a combination thereof. The metal can include iron and also include manganese, nickel, chromium, molybdenum, boron, titanium, vanadium, tungsten, cobalt, niobium, or a combination thereof.

The method can include contacting the metal with the corrosion inhibition composition, or with the corrosion inhibition compound and/or a reaction product thereof, under any suitable conditions, such as neutral, acidic, or basic conditions. The method can include contacting the metal with the corrosion inhibition composition, or with the corrosion inhibition compound and/or a reaction product thereof, under acidic conditions. For example, the metal can include contacting the metal with an aqueous acidic solution that includes the compound and/or a reaction product thereof. The method can further include removing the metal from the solution including the compound and/or reaction product thereof, which can leave a residual coating and/or adsorbed layer of the compound and/or reaction product thereof on the surface of the metal, wherein the residual layer and/or adsorbed layer has a corrosion inhibiting effect on the metal. The method can include rinsing the metal after removal from the solution including the compound and/or reaction product thereof, such as to remove residual solution from the surface of the metal while leaving a residual coating and/or absorbed layer from the compound and/or reaction product thereof intact on the surface of the metal.

The corrosion inhibiting effect can occur via any suitable mechanism. The corrosion inhibiting effect can be caused by the compound itself, a reaction product of the compound and the metal surface, a reaction product of the compound and acid, a reaction product of the compound and acid and the metal, or a combination thereof. The corrosion inhibiting effect can be provided during acidic conditions used to expose the compound and/or reaction product thereof to the surface of the metal. The corrosion inhibiting effect can be provided after exposure to acidic conditions used to expose the compound and/or reaction product thereof to the surface of the metal (e.g., via a residual coating and/or absorbed layer from the compound and/or reaction product thereof on the surface of the metal.

### Metal Having a Decreased Rate of Corrosion.

Various aspects of the present disclosure provide a metal having a decrease rate of corrosion. The metal can include a coating on an exterior surface thereof provided via contacting with the corrosion inhibition composition of the present disclosure, or with the corrosion inhibition compound of the present disclosure and/or a reaction product thereof. The coating can include an adsorbed layer on an exterior surface thereof provided via contacting with the corrosion inhibition composition of the present disclosure, or with the corrosion inhibition compound of the present disclosure and/or a reaction product thereof. The reaction product can be a reaction product of the corrosion inhibition compound and any suitable one or more materials, such as (a) an acid, or (b) the exterior surface of the metal, or (c) a combination thereof. The metal can be any suitable metal that can experience corrosion in the absence of the corrosion inhibition compound or reaction product thereof. For example, the metal can include aluminum, copper, iron, zinc, manganese, nickel, chromium, molybdenum, boron, titanium, vanadium, tungsten, cobalt, niobium, an alloy thereof, or a combination thereof. The metal can include aluminum, copper, iron, zinc, an alloy thereof, or a combination thereof. The metal can be a steel, such as a carbon steel, an alloy steel, or a combination thereof. The metal can include iron and also include manganese, nickel, chromium, molybdenum, boron, titanium, vanadium, tungsten, cobalt, niobium, or a combination thereof.

### Method of Forming Corrosion Inhibition Compound.

Various aspects of the present disclosure provide a method of forming the corrosion inhibition compound of the present disclosure. The method can be any suitable method that generates the corrosion inhibition compound. The method can include reacting a substituted or unsubstituted indole-3-carboxaldehyde with a substituted or unsubstituted thiosemicarbazide to form the corrosion inhibition compound. In various aspects, the indole-3-carboxaldehyde is naturally derived.

### EXAMPLES

Various aspects of the present invention can be better understood by reference to the following Examples which are offered by way of illustration. The present invention is not limited to the Examples given herein.

### Example 1. Synthesis and Characterization of Indole-3-Thiosemicarbazone Corrosion Inhibitors

### Experimental

Synthesis of the organic corrosion inhibitors. All starting chemicals were purchased from Sigma-Aldrich, Oakwood Chemical, and Alfa Aesar; solvents were purchased from Fisher Scientific and used without further purification. All reactions were carried out in flame-dried glassware under argon with dry solvents under anhydrous conditions unless otherwise stated. A solution of either indole-3-carboxaldehyde (0.4935 g; 3.4 mmol) or 5-methoxyindole-3-carboxaldehyde (0.5956 g; 3.4 mmol) was prepared in 22 mL of ethanol. Thiosemicarbazide 0.483 g (5.3 mmol) was subsequently added, and the solution was refluxed at 85° C. for 4 h. Once cooled the ethanol was removed by vacuum. The residue was then washed with 25 mL of 1 N HCl and 25 mL of deionized water, and dried in high vacuum. The indole-substituted inhibitors were synthesized using a slightly different method, starting with a solution of indole (2.343 g; 20 mmol) in dry dimethylformamide (DMF) to which 0.88 g of NaH (22 mmol) was added at 0° C. After stirring for 30 min at 0° C., 2.748 g (24 mmol) of 2-chloropyrimidine was added to the mixture. The solution was then heated under vigorous stirring to 130° C. and kept at this temperature for 24 h. The solution was then cooled to room temperature, poured into water, and extracted with ethyl acetate. The organic phase was then concentrated and purified by flash column chromatography. In a second step, the substituted indole was reacted with POCl₃ in DMF under argon at 0° C. The reaction mixture was slowly brought to room temperature and stirring was continued for another 2 h. Once completed, the reaction mixture was poured into cold saturated sodium bicarbonate and stirred for 30 min. A solid precipitated out and was collected by filtration. The solid was then washed with H₂O and hot hexane to afford the desired product with 90% yield.

All the organic corrosion inhibitors were purified either by recrystallization or flash column chromatography using silica gel 60 Å with a particle size of 0.032-0.063 mm. ¹H nuclear magnetic resonance (NMR) spectra of the recovered products were acquired in CDCl₃ or DMSO-d6 using a Varian MR-400 (400 MHz) or Bruker NEO 400 (400 MHz) spectrometer. High-resolution mass spectra (HRMS) were recorded on an Agilent 6540 QTOF (quadrupole time of flight) mass spectrometer using electrospray ionization. All yields refer to isolated products either by silica gel chromatography or by recrystallization.

Preparation of mild steel samples. Mild steel A366/1008 sheets (Fe—99.39%, Mn—0.38%, C—0.07%, Al—0.045%, Cr—0.024%, Ni—0.02%, others) were purchased from Online Metals and cut to 1 cm×3 cm×0.076 cm coupons. The coupons were manually polished with 600, 800, 1200, and 2000-grade sandpaper and subsequently sonicated in ethanol to remove any steel dust formed during the polishing process. After cleaning, the samples were wiped and placed in a drying oven at 60° C. for 30 min to evaporate any excess ethanol. Insulating tape was thoroughly placed on the front and back sides of the dried coupons to decrease the exposed metal surfaces to squares of 1 cm² each. The active corrosive surface area, including all sides, was 2.228 cm².

Electrochemical analysis. Open circuit voltammetry (OCV), electrochemical impedance spectroscopy (EIS), and potentiodynamic polarization were employed to characterize the corrosion behavior of the mild steel coupons in 0.1 M H₂SO₄ (Sigma Aldrich, research grade). A single-compartment 3-electrode system was utilized, with mild steel coupons acting as the working electrode, a platinum coil as the counter electrode, and an Ag/AgCl electrode saturated in 4 M KCl as the reference electrode (Pine Research Instrumentation). The electrolyte was freshly prepared before each experiment by dissolving 1 mM of organic inhibitor in 50 mL of 0.1 M H₂SO₄ using a tip sonicator for 15 min (Branson 450 Digital Sonifier with a tip size of 0.5 inches and operated at 50% amplitude). All the electrochemical measurements were performed using a Biologic VSP 300 potentiostat equipped with the EC-LAB software. Prior to every measurement, the electrolyte was purged with Argon for 30 min to remove any dissolved oxygen from the solution, and an Ar atmosphere was maintained throughout the analysis to ensure inert conditions. The electrochemical cell was placed in a water bath at 30° C. and maintained at this temperature throughout the experiment. OCV measurements were initially conducted for 30 min to stabilize the system, followed by EIS measurements in the frequency range of 0.1 MHz to 0.1 Hz using an amplitude of 10 mV at open circuit potential. Finally, potentiodynamic polarization measurements were performed in the range of ±500 mV of open circuit potential, with a scan rate of 50 mV s⁻¹.

Gravimetric analysis. Gravimetric tests were performed to study the weight change of mild steel coupons during the corrosion process in the presence and absence of organic corrosion inhibitors. Mild steel coupons were completely immersed in 0.1 M H₂SO₄ for 3 h at various temperatures (30, 35, 40, 45, and 50° C.) and different concentrations of corrosion inhibitor (1, 0.8, 0.6, 0.4. 0.2, and 0 mM). A water bath was used to maintain a constant temperature throughout the experiment. The weights of these coupons were measured before and after corrosion, after drying using a stream of dry air and gentle patting of the surface using a dust-free Kimwipe cloth (Kimtech, Kimberly-Clark) to remove any residual solution from the surface.

Scanning electron microscopy. Scanning electron microscopy (SEM) images and energy-dispersive X-ray spectra (EDS) were acquired using a FEI Quanta 250 field-emission microscope equipped with an Oxford Aztec™ energy-dispersive spectrometer. SEM images of polished mild steel coupons were recorded prior to the corrosion process while taking note of the axial position of the area of interest. These mild steel coupons were then subjected to corrosion in the presence and absence of 1 mM inhibitor in 0.1 M H₂SO₄ for a duration of 1 h at 30° C. The corroded coupons were dried using a stream of air and gently wiped to eliminate residual acid on the surface. Finally, SEM images of the corroded coupons were recorded at the previously noted axial position to study the changes in surface morphology of the mild steel coupons with and without inhibitors. EDS spectra were recorded for selected areas to determine the local and average elemental compositions of the surface.

Mass spectrometry analysis and imaging. Mild steel coupons corroded in the presence of inhibitors and reference coupons (an uncorroded coupon and a coupon corroded in blank H₂SO₄) were submitted to the Iowa State University W.M. Keck Metabolomics Research Laboratory (RRID:SCR 017911) for LC-MS analysis. These coupons were placed in 20 mL glass tubes with Teflon-lined caps. A liquid extraction was initiated with the addition of 6 mL of 1:1:1 LC-MS grade methanol:water:acetonitrile (Fisher Scientific) for mild steel coupons corroded in IT-1, IT-2, and IT-3. For IT-4, 18 mL of 1:1:7 methanol:20 mM HCl:acetonitrile was used for the extraction solvent. Samples were then vortexed for 10 s and sonicated for 10 min in a sonicating water bath (Model 2510, Branson Ultrasonics). The vortex and sonication steps were repeated three times before the sample extracts were allowed to settle, and the extract supernatants were recovered and transferred to glass autosampler vials. Similarly, neat coupons were treated using the same extraction solvents. The neat standards were prepared as 5 μg mL⁻¹ samples for the initial LC-MS profiling. Additionally, standard curves were prepared for each inhibitor molecule, with amounts ranging from 0.33 to 133 μg per sample. After sample preparation, the sample extracts were immediately subjected to LC-MS analysis.

For imaging, mild steel coupons (uncorroded, corroded in blank H₂SO₄, corroded in the presence of inhibitor) were coated with α-cyano-4-hydroxycinnamic acid (CHCA; Sigma-Aldrich) as the matrix, which was applied using an oscillating capillary nebulizer (OCN) sprayer. In short, three mild steel coupons were coated in sets of three plates at a time for three spray deposition cycles of 300 μL of 10 mg mL⁻¹ CHCA in 7:3 acetonitrile:water with 1% trifluoroacetic acid; all solvents were LC-MS grade (Fisher Scientific). The coupon positions were rotated after each deposition cycle to ensure even matrix deposition. The OCN unit was operated with a matrix solution flow rate of 2 mL h⁻¹ and a nitrogen gas pressure of 70 kPa with a flow rate of 4.5 L min⁻¹. After the matrix deposition, the coupons were dried in a vacuum desiccator for 30 min. Images of the prepared samples were acquired using a Bruker SolariX Fourier-transform ion cyclotron resonance mass spectrometer (FT-ICR MS) equipped for matrix-assisted laser desorption ionization (MALDI).

Theoretical calculations. The molecular properties of the four indole thiosemicarbazone inhibitors were computed in both vacuum and in water solvated models using the Gaussian 16 software. The calculations were done using density functional theory (DFT) at the B3LYP hybrid functional level and the 6-31G (2df, p). Water solvation was modeled implicitly by employing the self-consistent reaction field (SCRF) module and the Onsager model reaction field calculation consisting of the dipole polarizable continuum model (DPCM) method that was incorporated within the Gaussian 16 code, where the solute molecule was placed in a cavity within the solvent reaction field.

Using both vacuum and solvated models, the following theoretical parameters were calculated for the aforementioned molecules: 1) highest occupied molecular orbital (HOMO), 2) lowest unoccupied molecular orbitals (LUMO), 3) the Mulliken charges of these two molecular levels, 4) the bandgap that is the energy difference between HOMO and LUMO orbitals, 5) absolute electronegativity, 6) global hardness, 7) global softness, and 8) the fraction of electrons transferred from each molecule to the metal surface. The amount of the electron transferred was calculated by assuming the electronegativity of the iron metal surface (χ_{Fe}=7.0 eV), as reported in the literature.

Furthermore, ab initio molecular dynamics (AIMD) simulations were performed for molecules IT-1 and IT-2 to understand the adsorption mechanisms and energetics of the inhibitor molecules on the Fe surface. Molecules IT-1 and IT-2 were selected based on their higher performance than inhibitors IT-3 and IT-4.

The calculations were performed for the Fe(110) surface as it is one of the most dominant iron surfaces, it has the lowest surface energy, and most studies on organic corrosion inhibitors focused on their adsorption on the FE (110) facet. An optimized Fe unit cell was extended to a 4×4×2 in the x-, y-, and z-directions, respectively, to create a supercell. The supercell was then rotated 900 in the xy-direction to create a surface area of 16.06 Å² in size, and was then cleaved in the z-direction to create the Fe (110) slab. The Fe unit cell, bulk, and the (110) slab were all optimized with the convergence criteria of 1×10⁵ eV for electronic self-consistent iterations, until all the forces acting on ions were less than 0.02 eV/A. The 3×3×3 and 3×3×1 meshes of k-points in the Monkhorst-Pack scheme were chosen for the Brillouin zone sampling of the bulk and the slab, respectively, along with the application of 0.1 eV of Gaussian smearing.

The adsorption of IT-1 and IT-2 inhibitors onto the iron surface were probed using AIMD simulations that were done at F-point sampling with a time step of δt=1 fs for at least 10 ps until all the forces converged. A cut-off of 400 eV was also employed, with the electronic energy convergence criterion of 1×10⁻⁴ eV that was enforced at each time step. The Nosé-Hoover algorithm was employed to maintain the average temperature at 300 K. In all cases, one molecule of inhibitor was put in the box, surrounded by water molecules. The effect of water on the adsorption of the inhibitors and its corresponding adsorption-free energy barriers was studied by employing two different models: 1) a water monolayer on top of the iron surface, and 2) bulk water. For the monolayer, the Fe (110) slab was fitted into a simulation cell of 16.06×16.06×30.68 Å³ subject to periodic boundary conditions. The z-length of the simulation box leaves ˜26.5 Å of a vacuum gap that was incorporated to avoid spurious interactions between adjacent slabs. The monolayer was included of 30 water molecules that were first absorbed on the Fe (110) surface to provide a complete monolayer coverage to the slab. The density of the water monolayer in the simulation box was approximately equivalent to ρ=0.13 g/cm³. Meanwhile, in the case of the bulk water, the size of the simulation was reduced to 16.06×16.06×20 Å³, which was filled with 140 water molecules to mimic the density of the water in real life that is ρ=1 g/cm³.

Avogadro and Visual Molecular Dynamics (VMD) were used to visualize the HOMO and LUMO orbitals, and for molecular dynamics trajectories analysis, respectively.

### Results and Discussion

Introduction. Here, we designed a new generation of corrosion inhibitors derived from indole-3-carboxaldehyde, a compound commonly found in human and animal digestive systems resulting from the bacterial degradation of L-tryptophan, one of the most prevalent dietary amino acids. We leveraged the indole structure as a platform that we derivatized to generate a set of structurally-similar molecules.

To enhance the performance of the bio-sourced indole, its aldehyde function was leveraged to couple thiosemicarbazones, a low-cost and widely available class of organosulfur compounds that find applications as metal ligands in coordination chemistry and as a safe family of antitumoral and antiparasitic drugs in medicine. The structural diversity of thiosemicarbazones, specifically the possibility to tune their side groups, enabled us to synthesize a wide range of indole derivatives using an elegant and environmentally benign 2-step synthesis. The obtained compounds were subsequently evaluated as corrosion inhibitors for mild steel using various electroanalytical, gravimetric, and spectroscopic techniques. Corrosion inhibitions as high as 98.9% were achieved, surpassing the performance of the best organic inhibitors reported to date (Table 1). Gravimetric tests and Langmuir isotherms further substantiated the electroanalytical findings, revealing adsorption Gibbs free energies exceeding −40 kJ/mol. The results underscored that chemisorption is the dominant mechanism governing the interaction between the mild steel surface and the designed inhibitor molecules. Density functional theory (DFT) and ab-initio molecular dynamics (AIMD) calculations highlighted the dominant parameters that control the inhibitors' performance as well as the role of solvation on the electronic structure of the organic molecules.

TABLE 1
Performance of some of the best organic corrosion inhibitors reported to date and
of the best indole thiosemicarbazone inhibitor synthesized in the present work.

| | | | Inhibition | |
|---|---|---|---|---|
| | | Corrosion | efficiency | |
| Corrosion Inhibitor | Substrate | medium | (% η_{R}) | Ref |
| Loquat leaves extract | Mild steel | 0.5M H₂SO₄ | 94.3 | Comparative |
| 2-(1-(4-((Furan-2- | N80 steel | 4M HCl | 95.5 | Comparative |
| ylmethylene)amino)-5-thioxo- | | | | |
| 4,5-dihydro-1H-1,2,4-triazol- | | | | |
| 3-yl)-2-(1H-imidazol-2-yl) | | | | |
| ethyl)isoindoline-1,3-dione | | | | |
| 2-Hydroxyethyl-trimethyl- | Mild steel | 1M HCl | 96.0 | Comparative |
| ammonium acetate | | | | |
| 4-Ethoxybenzaldehyde | carbon steel | 1M HCl | 93.0 | Comparative |
| thiosemicarbazone | AISI 1020 | | | |
| 4-((1H-1,2,4-Triazol-3-yl) | Mild steel | 0.1M | 97.0 | Comparative |
| thio)-2H-chromen-2-one | A366/1008 | H₂SO₄ | | |
| Imidazolium Gemini Monomer | A3 carbon | 0.1M HCl | 96.6 | Comparative |
| | steel | | | |
| 1,3-bis-(5H-[1,2,4] | N80 steel | 4M HCl | 98.0 | Comparative |
| triazino[5,6-b]indol-3-yl) | | | | |
| thiourea | | | | |
| 7,9-ditetradecyl-6-methyl-7H- | Carbon steel | 1M HCl | 97.8 | Comparative |
| 1,2,4-triazolo[4,3-b][1,2,4] | | | | |
| triazepin-8-one | | | | |
| 4,6-bis(4-methoxyphenyl) | N80 steel | 4M HCl | 96.9 | Comparative |
| pyrimidin-2-amine | | | | |
| 2,2′-(propane-1,3-diylbis(2- | Mild steel | 1M | 93.0 | Comparative |
| oxoindoline-1-yl-3-ylidene)) | | H₂SO₄ | | |
| bis(hydrazine-1- | | | | |
| carbothioamide (II) | | | | |
| 2-((4-(3-methoxypropoxy)-3- | Mild steel | 0.5M | 95.1 | Comparative |
| methylpyridine-2-yl)- | | H₂SO₄ | | |
| methylthio)-1H-benzimidazole | | | | |
| 3,3′-(Pentane-2,4-diylidenebis | Mild steel | 1M | 96.4 | Comparative |
| (azanylylidene))-bis(N,N- | | H₂SO₄ | | |
| dimethyl-N-(2- | | | | |
| (hexadecyloxy)2-oxoethyl) | | | | |
| propan-1-aminium)chloride | | | | |
| 5-((4,5-Dihydrothiazol-2-yl) | Mild steel | 1M HCl | 97.4 | Comparative |
| methyl)quinolin-8-ol | | | | |
| (E)-2-((1H-Indol-3-yl) | Mild steel | 0.1M | 98.9 | Present work |
| methylene)hydrazine-1- | A366/1008 | H₂SO₄ | | |
| carbothioamide | | | | |

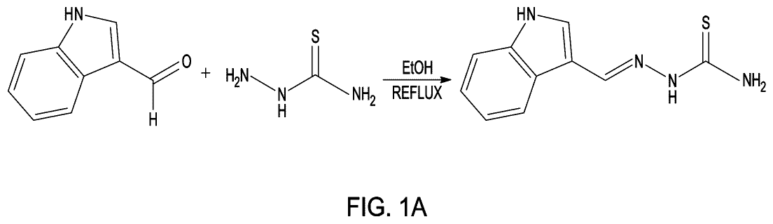

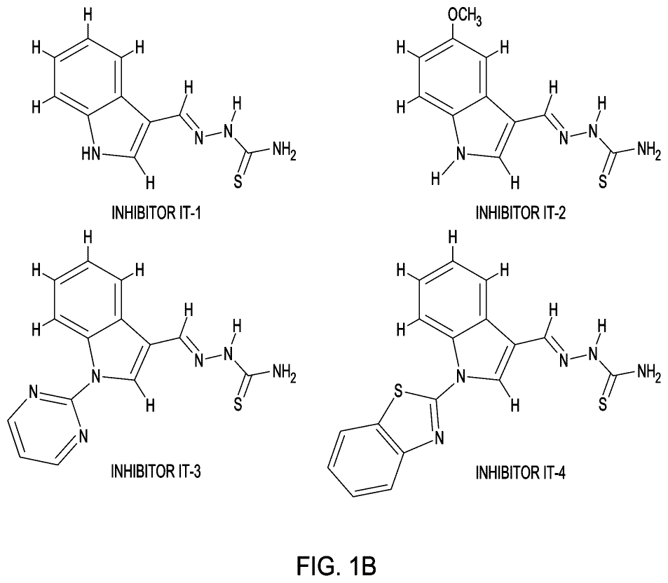

Synthesis and characterization. The corrosion inhibitors were synthesized using a simple and green procedure that leverages indole-3-carboxaldehyde, a product of the microbial degradation of L-tryptophan, and thiosemicarbazide, a safe and widely available organic compound key to chelating agents, and to antitumoral and antiparasitic drugs (FIG. 1A). Upon purification, the indole thiosemicarbazone (IT) inhibitors IT-1, IT-2, IT-3, and IT-4 were obtained in 70%, 96%, 75%, and 69% yield, respectively (FIG. 1B).

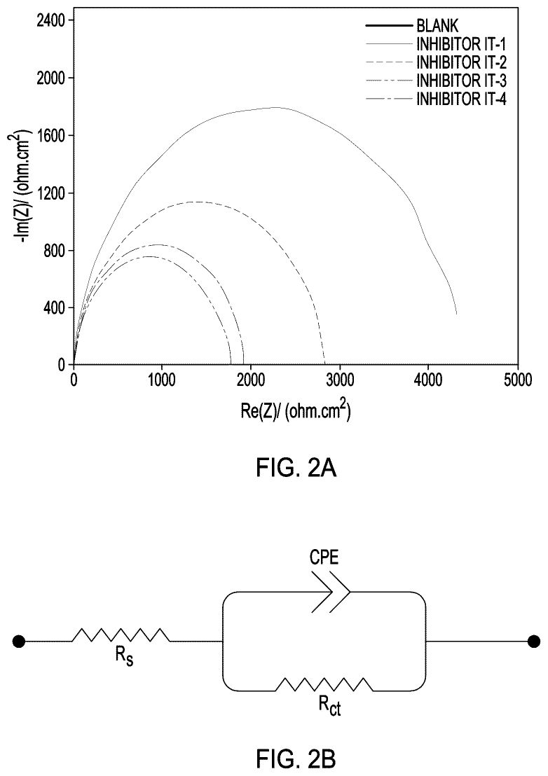

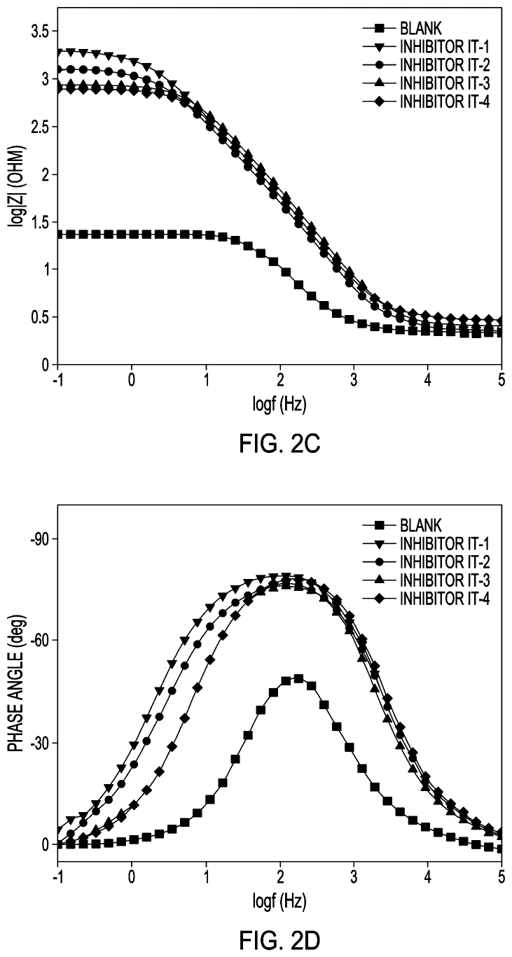

Electrochemical impedance spectroscopy. The electrochemical impedance of the mild steel coupons in the presence of inhibitor molecules was calculated using the Nyquist plots shown in FIG. 2A (mild steel in 0.1 M H₂SO₄ in the absence and presence of 1 mM of IT corrosion inhibitors). The fitting of the plots was done by approximating Randell's circuit, which consists of solution resistance (Rₛ) in series with a parallel arrangement of charge transfer resistance (R_{ct}) and constant phase element (CPE) as indicated in FIG. 2B. The corrosion parameters such as R_{ct}, double layer capacitance (C_{dl}), and inhibition efficiency (% η_{R}) were calculated from the equivalent circuit using Equations (1) and (2). The corresponding values are presented in Table 2.

$$\% {η}_{R} = \frac{{R}_{c t ( i )} - {R}_{ct ( o )}}{{R}_{c t ( i )}} \times 100 \qquad ( 1 )$$

$${C}_{d l} = {( {Y}_{0} {R}_{ct}^{1 - n} )}^{1 / n} \qquad ( 2 )$$

TABLE 2
Corrosion parameters calculated after fitting
the Nyquist plot to Randell's circuit.

| Inhibitor | Rₛ (Ω cm²) | R_{ct} (Ω cm²) | C_{dl} (μF/cm²) | % η_{R} |
|---|---|---|---|---|
| Blank | 4.9 | 47.2 | 210.48 | — |
| IT-1 | 5.0 | 4255.5 | 40.62 | 98.89 |
| IT-2 | 5.1 | 2782.8 | 45.40 | 98.30 |
| IT-3 | 6.7 | 1736.3 | 38.06 | 97.28 |
| IT-4 | 5.7 | 1903.2 | 32.82 | 97.52 |

From the data in Table 2, it can be observed that the charge transfer resistance (R_{ct}) increased by approximately two orders of magnitude in the presence of the inhibitors compared to the blank sample. This increase in the R_{ct} values indicates the formation of a protective layer on the surface of the mild steel coupon, which restricts the charge transfer between the surface and electrolyte, thereby preventing corrosion. In addition, it can be noted that the values of C_{dl} decreased in the presence of inhibitors, which is due to the resulting displacement of water molecules by the organic compounds on the surface of mild steel. The highest corrosion inhibition efficiency (%)R) was observed to be 98.89% for inhibitor IT-1. The % r)R values for inhibitors IT-2, IT-3, and IT-4 were found to be 98.30%, 97.28%, and 97.52%, respectively. Interestingly, the % r)R values for inhibitors IT-1 and IT-2 were higher than that for IT-3 and IT-4 despite the similar molecular structures of the four compounds.

The bode impedance plot presented in FIG. 2C showed a clear increase in the values of interfacial impedance at all frequencies in the presence of inhibitor molecules compared to the blank sample. This further supports that the inhibitor molecules are forming a protective layer over the surface of the mild steel. The bode phase angle plot (FIG. 2D) revealed that all the curves display a single-phase maximum, signifying a single time constant for the metal-acid interface, which can be assigned to the charge transfer process. The increase in the numerical value of the phase angle in the presence of an inhibitor is due to an increase in capacitive characteristics because of the formation of a protection layer over the mild steel surface, which acts as an electric double layer.

Potentiodynamic polarization curves. Polarization curves were used to calculate the corrosion parameters such as corrosion current density (I_{corr}), corrosion potential (E_{corr}), Tafel slopes (βₐ and β_{c}), and inhibition efficiency (% ηₜ). The values of I_{corr} and E_{corr} were determined with the aid of EC-LAB software using Tafel plots and % η_{I} was calculated from these I_{corr} values by using the following Equation (3):

$$\% {η}_{I} = \frac{{I}_{o} - {I}_{i}}{{I}_{o}} \times 100 \qquad ( 3 )$$

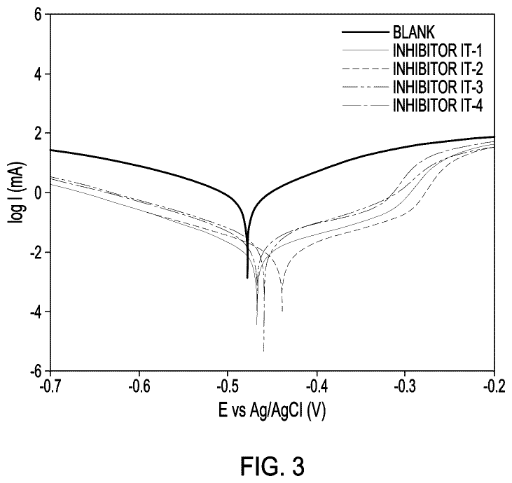

It can be observed from the polarization curves presented in FIG. 3 that both cathodic and anodic currents decreased in the presence of inhibitor molecules compared to the blank H₂SO₄ solution. This observation suggests that the inhibitor molecules are effective in mitigating both the hydrogen evolution reaction (HER) at the cathode and mild steel degradation at the anode, thus suppressing corrosion. Furthermore, Table 3 indicates that the E_{corr} values shifted marginally towards anodic potentials in the presence of the inhibitors, with the most significant shift of 38.6 mV observed for inhibitor IT-2. Since this shift falls within the range of ±85 mV, it can be inferred that the inhibitor molecules protect both cathodic and anodic sites from corrosion, thereby exhibiting mixed behavior.

TABLE 3
Corrosion parameters calculated from polarization curves performed
on mild steel coupons with/without inhibitor molecules.

| | E_{corr} | I_{corr} | βₐ | β_{c} | |
|---|---|---|---|---|---|
| Inhibitor | (mV) | (μA cm⁻²) | (mV/dec) | (mV/dec) | % η_{I} |
| Blank | −478.0 | 486.27 | 120.1 | 147.7 | — |
| IT-1 | −467.6 | 6.78 | 127.4 | 107.3 | 98.6 |
| IT-2 | −439.4 | 5.82 | 127.1 | 121.8 | 98.8 |
| IT-3 | −459.2 | 15.51 | 121.8 | 118.4 | 96.8 |
| IT-4 | −467.2 | 18.44 | 167.6 | 123 | 96.2 |

From the data presented in Table 3, it is evident that the highest current density was observed in the absence of the inhibitor, at 486.27 μA cm⁻². The corrosion current decreased in the presence of an inhibitor because corrosion involves the transfer of electrons between mild steel and the acidic solution. The inhibitor molecules protect the mild steel by impeding this electron transfer, thereby mitigating corrosion. It is observed that in the presence of inhibitor molecules, the corrosion currents significantly decreased compared to the blank sample, with the lowest corrosion current observed for inhibitor IT-2 at 5.82 μA cm⁻². From the % ηₜ values, it can be inferred that inhibitors IT-1 and IT-2 exhibit better inhibition properties compared to inhibitors IT-3 and IT-4. Furthermore, the anodic Tafel slope (βₐ) values remain almost constant (except for inhibitor IT-4), suggesting that the inhibitor molecules are adsorbed onto the mild steel surface at the initial stage of its corrosion, blocking surface sites from corrosion without altering the corrosion reaction mechanism.

Gravimetric analysis. The gravimetric analysis experiments were performed for different concentrations of inhibitor and at different temperatures. Corrosion parameters such as surface coverage (θ), corrosion rate (Ø), and inhibition efficiency (% η_{G}) were calculated using the following Equations (4)-(6), where wₒ is weight loss in the absence of inhibitor, wᵢ is the weight loss in the presence of inhibitor, D is the density of mild steel coupons (g cm⁻³), A is the surface area of the mild steel coupon (cm²), t is the time length of the experiment (h):

$$θ = \frac{{w}_{o} - {w}_{i}}{{w}_{o}} \qquad ( 4 )$$

$$Ø = \frac{87.6 \times ( {w}_{o} - {w}_{i} )}{D \times A \times t} \qquad ( 5 )$$

$$\% {η}_{G} = \frac{{w}_{o} - {w}_{i}}{{w}_{o}} \times 100 \qquad ( 6 )$$

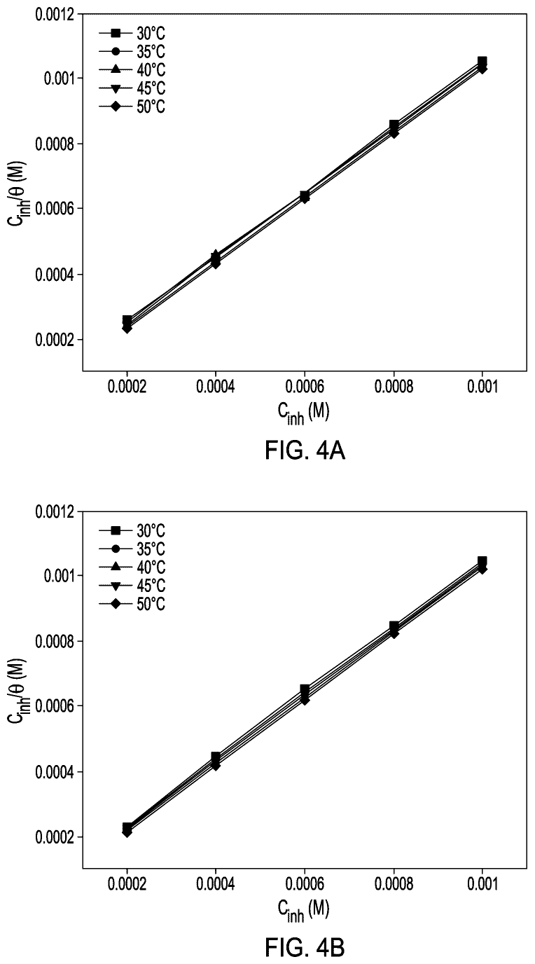

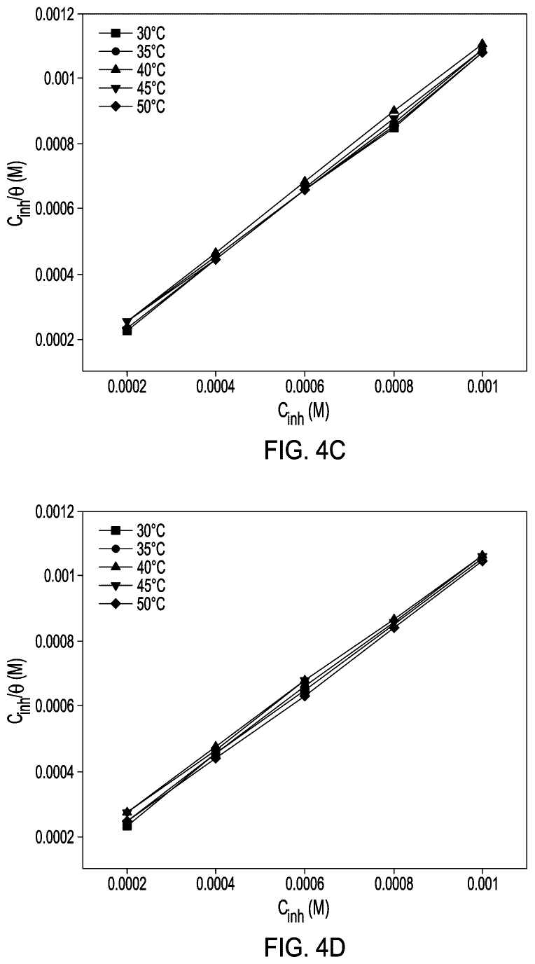

Langmuir adsorption isotherms. We have studied various models to analyze the adsorption characteristics of the corrosion inhibitors on the surface of mild steel. We found that Langmuir adsorption isotherms provide the best when analyzing the adsorption results obtained for the four inhibitors. The adsorption isotherms (FIGS. 4A-D) were generated by using the concentration of inhibitors (Cᵢₙₕ) used during gravimetric experiments and surface coverage (θ) by using the following linear expression (Equation (7)):

$$\frac{{C}_{i n h}}{θ} = \frac{1}{{K}_{ads}} + {C}_{i n h} \qquad ( 7 )$$

Experiments were performed at five different temperatures (30 to 50° C.), and the intercept obtained from these plots was used to calculate the equilibrium constant of adsorption (K_{ads}). The Gibbs free energy of adsorption (ΔG_{ads}) of the inhibitor molecules was calculated using the K_{ads} values using Equation (8), where T is the temperature (K), R is the universal gas constant (8.314 J K⁻¹ mol⁻¹), and 55.5 is the concentration of water (M) in the solution; these adsorption parameters were interpreted from FIGS. 4A-4D:

$$Δ {G}_{ads} = - RT \times ln ( 55.5 \times {K}_{ads} ) \qquad ( 8 )$$

The obtained equilibrium constants of adsorption K_{ads} were in order of 10¹ across all temperatures (Table 4). These values were on par with previously reported equilibrium constants and were consistent with the strong adsorption of the inhibitors to the surface of mild steel. IT-2 displayed the highest ΔG_{ads} (−41.44 kJ/mol) at 50° C., suggesting that inhibitor IT-2 is the most effective in mitigating mild steel corrosion. The order of K_{ads} and ΔG_{ads} is as follows: IT-2>IT-3>IT-1>IT-4. Additionally, ΔG_{ads} ranged between −34.9 to −41.4 kJ/mol. It has been reported that ΔG_{ads} values smaller than −20 kJ/mol indicate physisorption, while values larger than −40 kJ/mol suggest chemisorption. Since most ΔG_{ads} values are close to −40 kJ/mol, it can be inferred that the adsorption of inhibitor molecules on mild steel primarily occurs via chemisorption. This chemisorption likely involves the sharing of π-electrons of inhibitors with the unoccupied 3d orbitals of mild steel.

TABLE 4
The adsorption parameters calculated from Langmuir adsorption
isotherms for inhibitor molecules at temperature of 323K.

| Inhibitor | K_{ads} × 10⁴ (M⁻¹) | ΔG_{ads} (kJ/mol) |
|---|---|---|
| IT-1 | 3.16 | −38.63 |
| IT-2 | 9.00 | −41.44 |
| IT-3 | 4.76 | −39.73 |
| IT-4 | 2.38 | −37.86 |

Activation parameters. The calculation of the activation parameters, namely activation energy (Eₐ), activation enthalpy (ΔHₐ), and activation entropy (ΔSₐ), were performed using the corrosion rates (Ø) from gravimetric analysis and applying the Arrhenius Equation (9) and transition state Equation (10) which are presented below, where T is the temperature (K), R is the universal gas constant (8.314 J K⁻¹ mol⁻¹), λ is the pre-exponential factor, N_{A} is Avogadro's number=6.023×10²³ mol⁻¹, and h is Plank's constant=6.626×10⁻³⁴ m² kg s⁻¹.

$$log ( Ø ) = - \frac{{E}_{a}}{2.303 \times R T} + log λ \qquad ( 9 )$$

$$log ( \frac{Ø}{T} ) = log ( \frac{R}{{N}_{A} h} ) + ( \frac{Δ {S}_{α}}{2.303 \times R} ) - ( \frac{Δ {H}_{a}}{2.3 \; 03 \times RT} ) \qquad ( 10 )$$

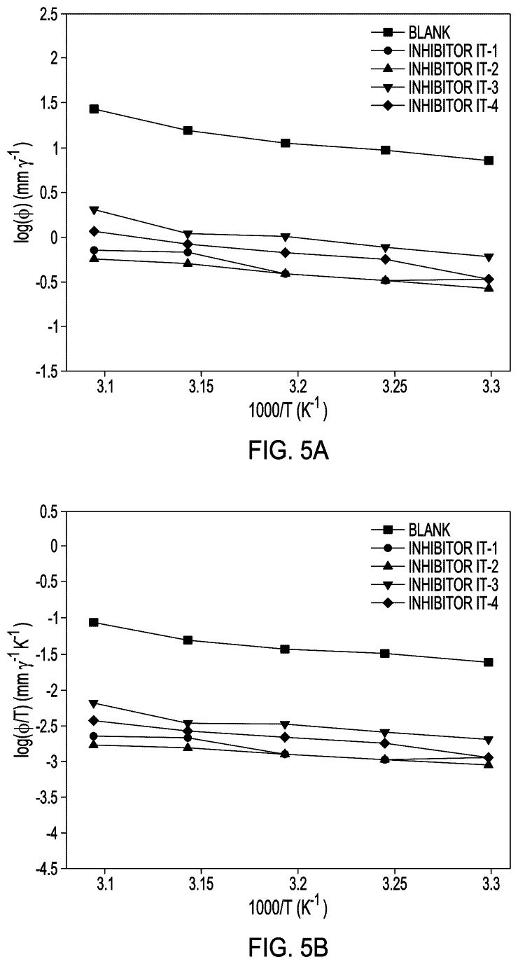

Activation energies were calculated for the adsorption process using the Arrhenius plot (log Ø vs. 1000/T) in FIG. 5A. Eₐ values decreased in the presence of inhibitors compared to the blank sample, likely due to the chemisorption of inhibitors by sharing π-electrons with the empty d-orbitals of mild steel (Table 5). It can also be seen that the values of Eₐ were in the order of: IT-2<IT-1<IT-3<IT-4. Hence, it can be inferred from the results that IT-1 and IT-2 display a better extent of adsorption on the surface of mild steel in comparison to inhibitors IT-3 and IT-4.

TABLE 5
Activation parameters estimated from Arrhenius
plots and transition state plots.

| Inhibitor | Eₐ (kJ/mol) | ΔHₐ (kJ/mol) | ΔSₐ (J/mol. K) |
|---|---|---|---|
| Blank | 50.94 | 48.34 | −69.88 |
| IT-1 | 36.33 | 33.73 | −143.96 |
| IT-2 | 29.20 | 26.60 | −167.97 |
| IT-3 | 44.32 | 41.72 | −111.94 |
| IT-4 | 47.50 | 44.90 | −105.37 |

Other activation parameters, such as ΔHₐ and ΔSₐ, were estimated using the transition state plot (log Ø/T vs. 1000/T) in FIG. 5B. From Table 5, it can be observed that the ΔHₐ values are positive, implying that corrosion of mild steel in 0.1 M H₂SO₄ is an endothermic process. The decrease in the ΔHₐ values in the presence of the ITs signifies the mitigation of the corrosion reaction by the inhibitor molecules. The order of ΔHₐ values is: IT-2<IT-1<IT-3<IT-4. The negative value of ΔSₐ for the blank sample results from the mechanism of HER occurring on the mild steel surface. The HER process includes a sluggish electron transfer from the mild steel substrate, followed by either chemical recombination at lower overpotential levels or electrochemical desorption reactions at higher overpotential levels. The decrease in randomness due to these intermediate steps results in the negative values of ΔSₐ. In the presence of inhibitor molecules, ΔSₐ values were even more negative compared to the blank sample, inferring that the activated states of these processes in the presence of the inhibitor became more organized. As the inhibitor effectively obstructs the cathodic reaction sites to a significant degree, the adsorption of protons on the metal surface and the recombination of surface-adsorbed hydrogen atoms necessitate closer interactions, leading to a more negative ΔSₐ value. The order of ΔSₐ values is as follows: IT-2<IT-1<IT-3<IT-4. This indicates that inhibitors IT-2 and IT-1 are better at adsorption onto the mild steel substrate and thus act as better inhibitors.

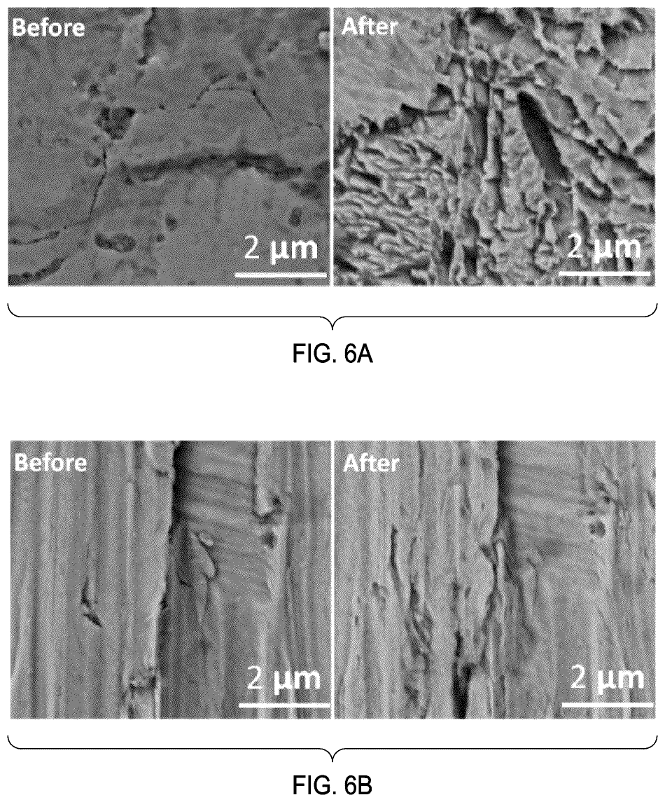

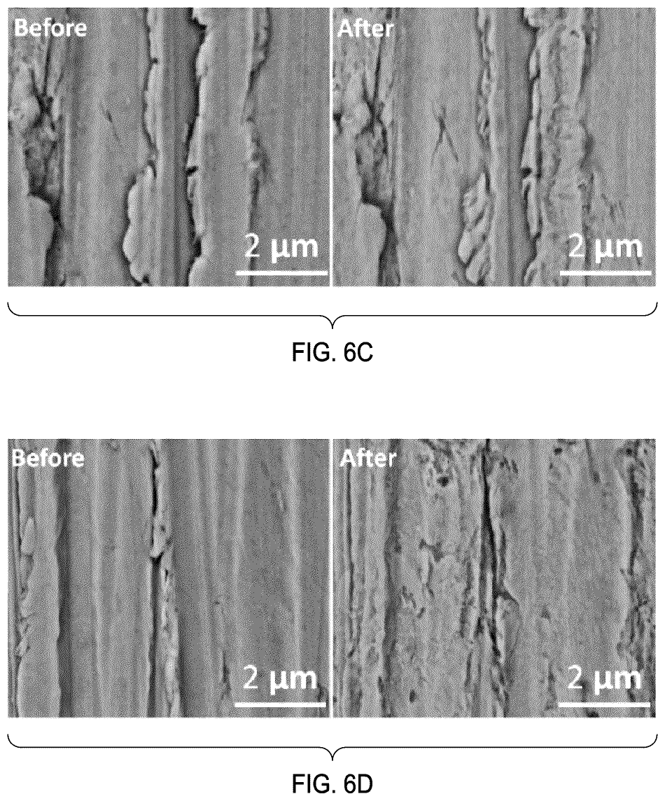

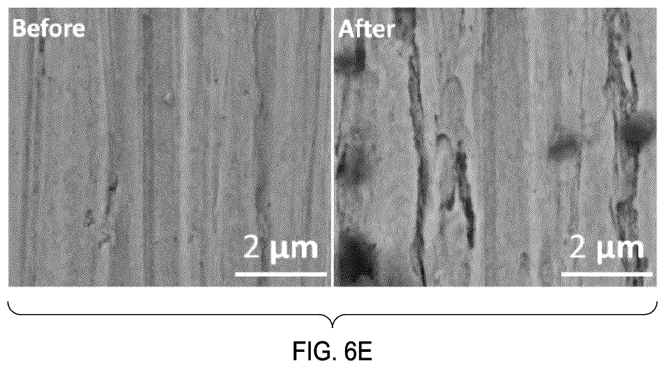

SEM and EDS analysis. SEM characterizations of the mild steel coupons were performed to investigate the effect of the organic inhibitors on surface topology during 1-hour corrosion tests in 0.1 M H₂SO₄. To ensure a thorough investigation, SEM images were acquired for the same areas before and after corrosion by taking note of the corresponding axial positions. A rough and unrecognizable surface was observed for the mild steel coupon tested in the absence of an inhibitor (FIG. 6A), consistent with the well-documented pitting and partial dissolution that takes place during the corrosion of mild steel in acidic media. In contrast, the mild steel's surface was well preserved when 1 mM of corrosion inhibitor was added to the acidic medium, with the same surface features being clearly visible before and after corrosion (FIGS. 6B-D). These SEM images visually support the electroanalytic and gravimetric results obtained under similar conditions and infer that the inhibitor molecules effectively protect the whole surface of the coupons from corrosion. They also confirm the higher performance of IT-1 and IT-2 as the corresponding images show fewer alterations than those recorded for IT-3 and IT-4.

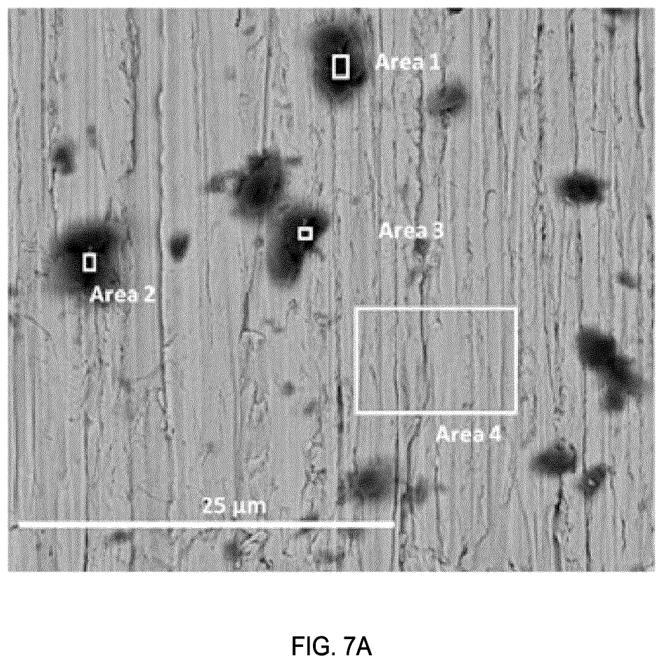

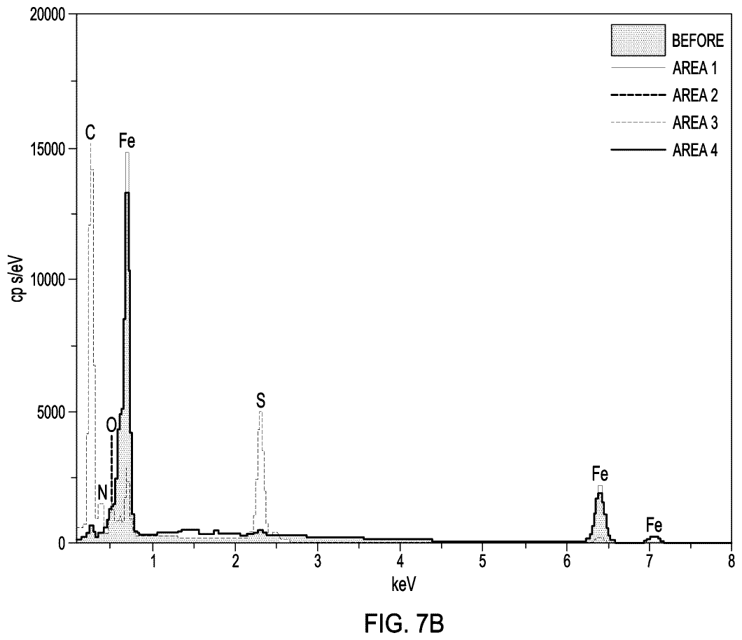

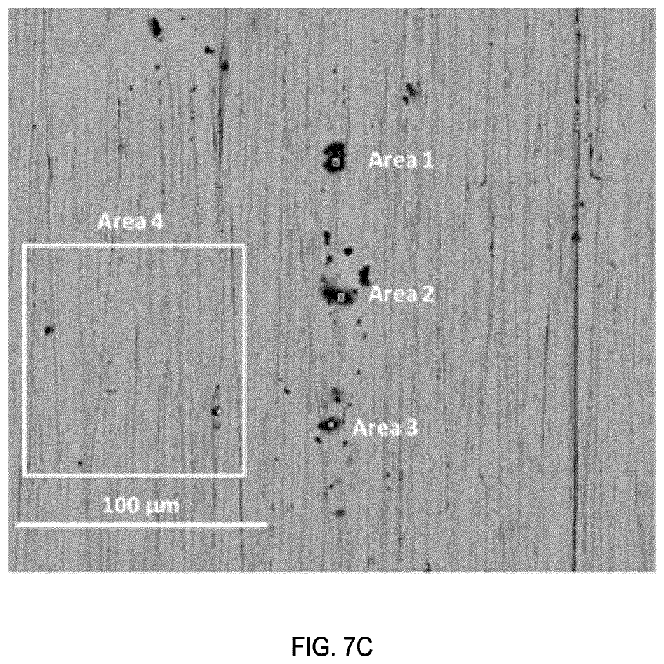

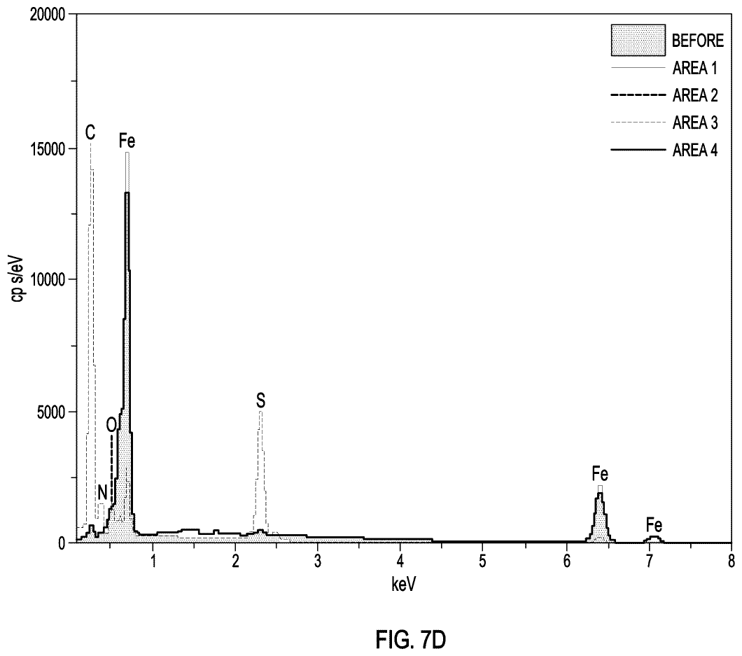

Interestingly, several dark spots were observed in the backscattered electron images for IT-4 (FIG. 6E), suggesting the presence of lower atomic weight elements. These areas were further investigated by EDS to determine the elemental composition of the dark regions and their possible origin. FIGS. 7C-7D and Table 6 revealed that the spots contain high concentrations of C, N, S, and a proportionally lower concentration of Fe compared to other areas of the coupon. Considering the analysis depth of EDS (1-2 μm), the elemental analysis suggests that the dark spots correspond to micron-sized deposits of IT-4 on the surface of mild steel, which suggests a lower solubility of this inhibitor in the acidic solution compared to the other compounds. Similar dark spots, albeit significantly smaller, were also found on the surface of the mild steel coupon treated with IT-3 (FIGS. 7A-7B). Collectively, these results may explain the deviations between electroanalytic and gravimetric data noted for IT-3 and IT-4 (vide infra).

TABLE 6
Elemental composition in weight % calculated from the EDS analysis
of selected areas of the mild steel coupons before and after corrosion in
0.1M H₂SO₄ in the presence of inhibitors IT-3 and IT-4. Areas 1-3
correspond to spot analyses of the dark regions visible in FIGS. 7A and
7C, while Areas 4 correspond to larger regions free of dark features
(white rectangles in FIGS. 7A and 7C).

| Label | C | N | O | S | Fe | Total |
|---|---|---|---|---|---|---|
| Inhibitor IT-3 | | | | | | |
| Before Corrosion | 1.56 | — | 0.65 | — | 97.70 | 100 |
| After corrosion Area 1 | 61.85 | 28.67 | — | 9.08 | 0.41 | 100 |
| After corrosion Area 2 | 60.40 | 23.89 | 0.85 | 10.27 | 4.58 | 100 |
| After corrosion Area 3 | 60.53 | 25.41 | — | 9.16 | 4.89 | 100 |
| After corrosion Area 4 | 1.94 | — | 1.48 | 0.74 | 95.85 | 100 |
| Inhibitor IT-4 | | | | | | |
| Before Corrosion | 1.24 | — | 0.65 | — | 98.11 | 100 |
| After corrosion Area 1 | 49.62 | 9.56 | 4.15 | 14.16 | 22.51 | 100 |
| After corrosion Area 2 | 48.88 | 8.11 | 3.60 | 14.12 | 25.29 | 100 |
| After corrosion Area 3 | 50.72 | 11.2 | 4.11 | 12.10 | 21.88 | 100 |
| After corrosion Area 4 | 1.66 | — | 1.02 | 0.53 | 96.80 | 100 |

XPS spectroscopy. The mild steel coupons were analyzed by XPS after exposure to corrosion media containing 1 mM of inhibitors. The C, O, N, and Fe spectra were deconvoluted. The CIs spectra exhibited peaks around 284.4 and 284.8 eV, consistent with the presence of C—C, C═C, and C—H functionalities in the inhibitor molecules. Peaks near 286.2 eV correspond to the C—N linkage within the inhibitor molecules. The presence of thioamide functional groups for all inhibitor molecules gave a peak at 287.5 eV. The peak at 288.7 eV is attributed to the interactions between carbon atoms in the inhibitor molecules and oxygen atoms from the oxidized mild steel surface.

The O1s spectra revealed peaks around 529.9, 530.9, and 531.6 eV, corresponding to the presence of FeO, FeOOH, and Fe(OH)₃, respectively. These findings indicate the presence of oxygenated corrosion products over the protected mild steel substrate, which is consistent with prior reports. The N1s spectra showed peaks at 398.2 and 399.4 eV, confirming the presence of C═N and —C═S functionalities, respectively, in the inhibitor molecules. The peak around 400.5 eV is characteristic of aromatic-N linkages in the inhibitor molecules, with increased intensity observed for inhibitors IT-3 and IT-4, in good agreement with their molecular structures. Additionally, the N—N linkage in thiosemicarbazone groups gave a peak around 401.8 eV in the NIs spectra. The Fe 2p spectra appear as a doublet for all inhibitor molecules, with 2p_{3/2} observed around 710.8 eV and 2p_{1/2} around 724.3 eV. The 2p_{3/2} peak is deconvoluted into three distinct peaks: one around 706.5 eV corresponding to the Fe⁰ state, one around 710.6 eV corresponding to the Fe²⁺ oxidation state, and one around 714 eV corresponding to the Fe³⁺ oxidation state. These results thereby indicate not only the presence of these inhibitor molecules over the surface of the mild steel coupon but also their attachment to the oxygenated moieties present on the surface of mild steel, thus forming a stronger interaction with the mild steel surface and protecting it from corrosion.

Mass spectrometry analysis and imaging. To quantify the amount of inhibitor molecules adsorbed onto the mild steel coupons, we conducted an LC-MS analysis of the metal samples after 1-hour tests in 0.1 M H₂SO₄ containing 1 mM of the desired inhibitor. Solutions containing known concentrations of inhibitors were also prepared and used as external standards for quantification. A list of molecular features was compiled for each inhibitor based on the observed features in the total ion chromatograms (TIC), making sure that the ions were present in abundance both in the standard solutions and in the solutions prepared using the corroded coupons. As can be seen in Table 7, the performance of the IT inhibitors did not scale with their surface concentrations. Relatively low amounts of IT-1 and IT-2, i.e., 5-13 μg per coupon, were sufficient to afford an inhibition efficiency of >98.5%. In contrast, the amounts measured for IT-3 and IT-4 were 1-2 orders of magnitude higher (80-176 μg per coupon). These findings are consistent with the SEM-EDS investigations and the gravimetric results, highlighting that a monolayer of a potent inhibitor is sufficient to offer good protection, while higher concentrations are usually the result of the inhibitor's lower solubility in the aqueous medium and/or the formation of agglomerates on the surface of mild steel. These results also confirm that the performance of an inhibitor is primarily controlled by its electronic structure and the free energy associated with its binding to the surface, not the amount of adsorbed inhibitor.

TABLE 7
Amounts (in μg) of indole thiosemicarbazone
inhibitors found by LC-MS on the surface of the
mild steel coupons after 1-hour corrosion tests.

| Inhibitor | Adsorbed weight (μg/plate) |
|---|---|
| IT-1 | 12.83 |
| IT-2 | 4.91 |
| IT-3 | 175.62 |
| IT-4 | 80.33 |

MALDI-MS was further performed on the coupons to confer spatial resolution to the LC-MS data and visualize the distribution of the inhibitors over the entire metal surface. The ions used for quantification by LC-MS served here to map the organic compounds adsorbed to the surface. Remarkably, the parent ions (M+H+, the protonated and positively charged molecular inhibitors with m/z of 390.10, 394.13, 297.09, and 320.09 for IT-1, IT-2, IT-3, and IT-4, respectively) were evenly distributed across the investigated areas and afforded the strongest signals relative to other ions. The resulting images confirmed that the 4 compounds bind to the entire metal surface, forming a protective layer and that the aggregates observed by SEM are not the result of poor metal-inhibitor interactions. IT-1 showed the most uniform and widespread coverage with virtually no fluctuation in local concentration. The calculated masses of the detected ions also suggest that the molecular integrity of the compounds is preserved during corrosion tests and that they do not experience significant amounts of acid- or metal-promoted degradation or transformation. This finding is important as it confirms the absence of artifacts and indicates that structure-activity relationships are within reach for this class of inhibitors

Theoretical calculations: HOMO and LUMO structures of the indole thiosemicarbazone inhibitors. The geometrical optimization of the inhibitor molecules was performed using DFT calculations to determine the electron-rich sites, i.e., highest occupied molecular orbitals (HOMO), and electron-deficient sites, i.e., lowest unoccupied molecular orbitals (LUMO). These calculations were performed for both the inhibitors in a vacuum and the solvated compounds to understand the effect of solvation on their performance.

The distribution of the HOMO and LUMO orbitals of the molecules in a vacuum was found to vary greatly depending on the nature of the side groups despite the four inhibitors sharing the same indole thiosemicarbazone platform structure. These differences highlight the importance of side groups on the electronic structure of the whole molecule, suggesting that the performance of an organic corrosion inhibitor can be tailored to a great extent through simple chemical derivatization. These results also highlight that the electronic structure of an organic corrosion inhibitor cannot be simply described as the cumulative contribution of the individual building blocks that form the molecule. As such, the presence of multiple electron-rich heteroatoms (N, S, O) is not a guarantee of high corrosion inhibition. Instead, these results suggest that DFT calculations are required to explain and predict their performance.

Our calculations also reveal the important role played by the solvent in the electronic structure and performance of these compounds. While the HOMO of IT-1 and IT-2 was localized in the thioamide part of the inhibitors when computed in vacuum, the orbital was also distributed in the indole part of the molecules upon solvation. Since HOMO orbitals are electron-rich, the redistribution of the HOMO for IT-1 and IT-2 indicates an enhanced electron donation capability of these inhibitor molecules under solvated conditions, where corrosion typically occurs. Interestingly, we observed minimal changes in the HOMO and LUMO distribution upon solvation for inhibitors IT-3 and IT-4.

In the simulated Mulliken charges for each of the atoms of the four inhibitors, as anticipated, the heteroatoms carry negative charges that facilitate the inhibitors' interactions with the electron-deficient metal surface. Interestingly, some of the charges became more pronounced for the solvated molecules, further highlighting the role of the solvent on their electronic structure and adsorption properties.

Theoretical calculations: Molecular reactivity calculations. The reactivity of organic corrosion inhibitors can be further estimated from the position of HOMO (E_{H}) and LUMO (E_{L}) orbitals, global hardness (γ), softness (σ), electronegativity (χ), and the fraction of electrons transferred to the surface of the metal (ΔN). These parameters were calculated for both the inhibitors in a vacuum and for the solvated molecules using the following Equations (11)-(15):

$$Δ E = {E}_{L} - {E}_{H} \qquad ( 11 )$$

$$χ = - \frac{{E}_{L} + {E}_{H}}{2} \qquad ( 12 )$$

$$γ = \frac{{E}_{L} - {E}_{H}}{2} \qquad ( 13 )$$

$$σ = 1 / γ \qquad ( 14 )$$

$$Δ N = \frac{{χ}_{Fe} - {χ}_{inh}}{2 {γ}_{inh}} ; {χ}_{F e} = 7. eV \qquad ( 15 )$$

The reactivity parameters calculated from the above equations are presented in Table 8. E_{H} values indicate the electron donation ability of the inhibitor molecule to the empty d-orbitals of metal atoms while E_{L} values indicate their electron back acceptance ability. It has been previously established that small values (less negative) for both E_{H} and E_{L} favor the strong adsorption of the organic corrosion inhibitors to the metal surface. Likewise, a lower ΔE, defined as the energy difference between HOMO and LUMO orbitals, is an indicator of chemical reactivity, which can play a role for inhibitors that protect metals through sacrificial oxidation. As can be seen in Table 8, IT-1 and IT-2 have 0.1-0.2 eV lower E_{H} and E_{L} energies than IT-3 and IT-4. The solvent further lowers their energies by 0.4-0.6 eV, in good agreement with the changes in the molecular orbitals and the stronger binding anticipated from the calculated HOMO and LUMO structures. Interestingly, the trend observed for ΔE values is inconsistent with the inhibition efficiencies observed experimentally. This discrepancy has been reported by other research groups and likely indicates that the IT inhibitors adsorb and form a protective layer on the metal rather than acting as a sacrificial redox compound.

TABLE 8
Molecular reactivity parameters obtained from DFT calculations.

| | E_{H} | E_{L} | ΔE | χ | γ | σ | |
|---|---|---|---|---|---|---|---|
| Inhibitor | (eV) | (eV) | (eV) | (eV) | (eV) | (eV⁻¹) | ΔN |
| IT-1 vacuum | −5.391 | −1.456 | 3.935 | 3.423 | 1.968 | 0.508 | 0.909 |
| IT-1 solvated | −4.939 | −0.814 | 4.125 | 2.876 | 2.062 | 0.485 | 1.000 |
| IT-2 vacuum | −5.285 | −1.381 | 3.904 | 3.333 | 1.952 | 0.512 | 0.939 |
| IT-2 solvated | −4.824 | −0.768 | 4.056 | 2.796 | 2.028 | 0.493 | 1.036 |
| IT-3 vacuum | −5.435 | −1.822 | 3.613 | 3.629 | 1.806 | 0.554 | 0.933 |
| IT-3 solvated | −5.025 | −1.204 | 3.114 | 3.114 | 1.910 | 0.523 | 1.017 |
| IT-4 vacuum | −5.428 | −1.832 | 3.630 | 3.630 | 1.798 | 0.556 | 0.937 |
| IT-4 solvated | −5.010 | −1.226 | 3.118 | 3.118 | 1.892 | 0.529 | 1.026 |

Other performance indicators, namely electronegativity, and the fraction of electrons transferred with the metal surface, are also fully consistent with the better performance of IT-1 and IT-2. Specifically, low electronegativity favors the transfer of electrons from the organic compound to the metal (χ_{Fe}=7.0 eV) until their chemical potentials come into equilibrium. The ˜0.3 eV lower values for IT-1 and IT-2 compared to IT-3 and IT-4 again support the stronger adsorption of the former. In addition, ΔN values lower than 3.6 imply a high tendency of the inhibitors to donate electrons to the mild steel substrate. Here, all the ΔN values were found to range between 0.9 and 1.0, indicating a similar adsorption process by electron donation for the four compounds.

Theoretical calculations: Ab-initio molecular dynamics (AIMD) calculations. AIMD calculations were performed to better understand the role played by the solvent at an experimentally relevant temperature of 300 K. The calculations were performed for the best-performing inhibitors, IT-1 and IT-2, in the presence of a water monolayer and bulk water to study (i) how the inhibitors displace the water molecules near the surface during adsorption, and (ii) how water alters their electronic structure and binding energy. All calculations were performed for Fe(110) as a representative surface for mild steel.

Very different interactions with the surface were observed for IT-1 and IT-2 in the presence of a water monolayer. IT-1 was found to physisorb on the water molecules through interactions between the indole fragment of the inhibitor and the hydrogen atoms of H₂O, while IT-2 did not bind under the studied conditions. However, spontaneous chemisorption occurred for both compounds in the presence of bulk water. These results further highlight that instead of impeding the diffusion and adsorption of the inhibitors, water molecules actually facilitate the chemisorption of the solvated compounds by promoting their electronic structure and binding affinity.

Surprisingly, AIMD calculations also predicted the spontaneous decomposition of IT-2 into three fragments that strongly bind to the surface and offer the same level of protection as the molecular inhibitor. This result is inconsistent with IT-2's ¹H NMR spectrum, LC-MS data, and XPS spectra (vide infra). As such this decomposition may either be pH dependent, or an artifact related to the limitations of AIMD calculations.

Conclusions. In conclusion, the biobased corrosion inhibitors developed in the present work demonstrate exceptional efficacy in mitigating mild steel corrosion in acidic environments. The findings from electrochemical impedance spectroscopy and polarization curve analyses indicated that they not only exhibit high corrosion inhibition efficiencies but also protect both cathodic and anodic sites from corrosion. Further, gravimetric analysis revealed that IT inhibitors follow the Langmuir model of adsorption, and the values of ΔG_{ads} (close to −40 kJ/mol) revealed they are chemisorbed to the surface of mild steel. From both electrochemical and gravimetric analyses, it can be inferred that inhibitors IT-1 and IT-2 have superior performance in mitigating corrosion in comparison to inhibitors IT-3 and IT-4.

SEM images of mild steel coupons before and after corrosion confirmed a significant reduction in corrosion in the presence of IT inhibitors. Additionally, inhibitors IT-3 and IT-4 were found to form deposits on the mild steel surface, which may account for the deviations observed between electrochemical and gravimetric analyses. LC-MS and MALDI-MS confirmed that the inhibitor molecules covered and protected the whole mild steel surface with IT-2 offering the highest protection at a surface concentration as low as 0.7 μg cm⁻². Moreover, DFT calculations provided valuable insights into the HOMO and LUMO positions on the molecules, revealing in particular the role of side groups on the electronic structure of the indole thiosemicarbazone platform. Other computed reactivity parameters aligned well with experimental observations. Overall, both experimental and theoretical results affirm that inhibitors IT-1 and IT-2 possess better corrosion inhibition capabilities compared to inhibitors IT-3 and IT-4 that can be traced back to their electronic structure. Finally, DFT and AIMD results showed that solvation played an important role in enhancing their electronic properties and driving their chemisorption.

### Example 2. Synthesis and Characterization of Indole-3-Thiosemicarbazone Corrosion Inhibitors

Additional indole-3-thiosemicarbazone corrosion inhibitors were synthesized and their corrosion inhibition activity was characterized. Any experimental procedures for synthesis and testing that are not described in this Example were performed in the same manner as described for Example 1.

Standard conditions were employed for determining the efficiency of corrosion inhibition using mild steel in 0.1 M sulfuric acid at 30° C. The corrosion inhibitors were dissolved in sulfuric acid at concentrations of 0.5 mM. Inhibition efficiencies were determined using electrochemical impedance spectroscopy (EIS). For EIS, a VSP-300 Potentiostat from Bio-logic with EC-LAB software was used. The test was performed in a conventional 3-electrode cell with an Ag/AgCl reference electrode, a Pt foil counter electrode, and a mild steel coupon as the working electrode. The counter electrode and working electrode were clamped using a PTFE holder sited in a PTFE cell head. A freshly made solution of 1 mM inhibitor molecule in either 0.1 M sulfuric acid or 0.3 M HCl (acid solution was made with nanopure water) was prepared before each test. Sonication for 30 min was generally used for molecules that were slow to dissolve. Mild steel A366/1008 (from Online Metals) was cut into 1 cm×3 cm coupon with a thickness of 0.076 cm. The steel was manually and sequentially polished with 600, 800, 1200, and 2000 grade sandpaper and then rinsed with ethanol and sonicated for 3 min. After wiping with a Kimwipe, the steel coupon was wrapped by parafilm to ensure that there was only a lower exposed surface area. The open circuit voltage was recorded and stabilized for 30 min. Electrochemical impedance measurements were performed at the open circuit voltage. Frequencies ranging from 10⁵ to 10⁻¹ Hz with an amplitude of 10 mV were used for the test. The potential was scanned from OCV−300 mV to OCV+300 mV with a 0.5 mV s⁻¹ sweep rate. After the test, the built-in software was used to display the Nyquist plot. The commercial organic corrosion inhibitor hexamethylenetetramine was also evaluated under the same conditions.

The following structure shows the chemical structure of corrosion inhibitors 1a-1d.

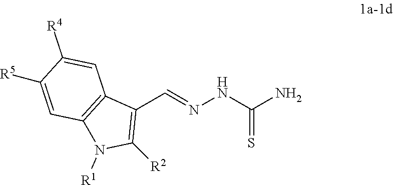

Table 9 summarizes the structures and corrosion inhibition data for compounds 1a-1d in sulfuric acid and HCl.

TABLE 9
Structures and inhibition efficiencies of compounds 1a-1d.

| Entry | R2 | R4 | R5 | R1 | IE H₂SO₄ | IE HCl | logP |
|---|---|---|---|---|---|---|---|
| 1a | H | H | OMe | H | 94 | 86 | 0.41 |
| 1b | H | Br | H | H | 91 | 89 | 1.36 |
| 1c | H | H | H | 2-thiazole | 94 | 89 | 2.45 |
| 1d | H | H | OH | H | 95 | 81 | 0.15 |

The following structure shows the chemical structure of corrosion inhibitor 2, wherein R═H and X=0, which had an IE of 96.6% in sulfuric acid and 89% in HCl.

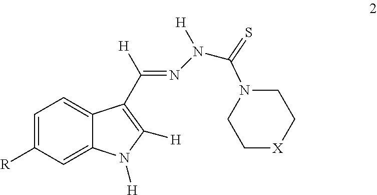

The following structure shows the chemical structure of corrosion inhibitor 3, wherein R═H, which also functioned well as a corrosion inhibitor, affording inhibition efficiencies of 97% in sulfuric acid and 86% in hydrochloric acid.

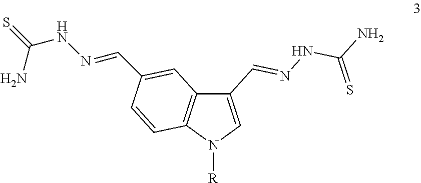

### Example 3. Synthesis and Characterization of Thiosemicarbazone Corrosion Inhibitors

Additional thiosemicarbazone corrosion inhibitors were synthesized and their corrosion inhibition activity was characterized. All experimental procedures for synthesis and testing were performed in the same manner as described for Example 1.

The following structure shows the chemical structure of corrosion inhibitors 1a, 1b, IT-5, and IT-6.

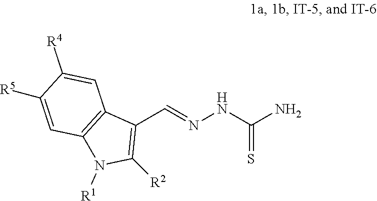

Table 10 summarizes the structures of compounds 1a, 1b, IT-5, and IT-6.

TABLE 10
Structures of compounds 1a, 1b, IT-5, and IT-6.

| Entry | R2 | R4 | R5 | R1 |
|---|---|---|---|---|
| 1a | H | H | OCH₃ | H |
| 1b | H | Br | H | H |
| IT-5 | H | OH | H | H |
| IT-6 | CH₃ | H | H | H |

Compound IT-7 had the following structure:

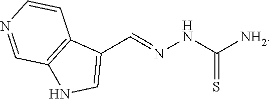 [SMILES: NC(=S)N/N=C/c1c[nH]c2cnccc12]

Compound IT-8 had the following structure:

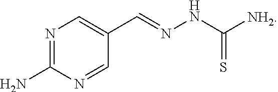 [SMILES: NC(=S)N/N=C/c1cnc(N)nc1]

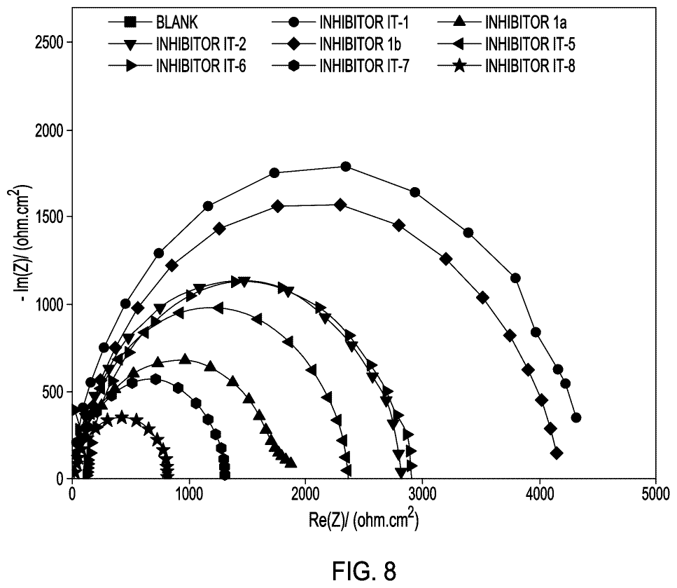

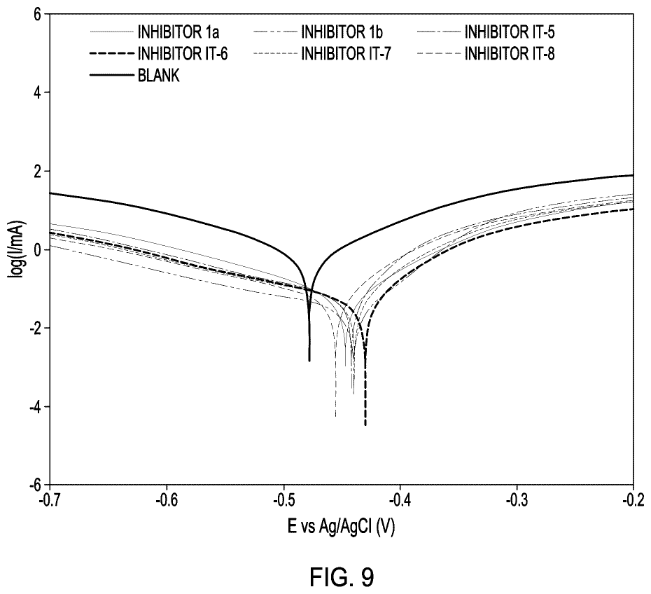

Table 11 gives electrochemical impedance spectroscopy data for compounds 1a, 1b, and IT-5-IT-8. FIG. 8 illustrates electrochemical impedance spectroscopy data for compounds IT-1, 1a, IT-2, 1b, and IT-5-IT-8.

TABLE 11
Electrochemical impedance spectroscopy data
for compounds 1a, 1b, and IT-5-IT-8.

| Inhibitor | R_{ct} (Ω cm²) | % η_{R} |
|---|---|---|
| Blank | 47.2 | — |
| 1a | 1746.9 (±0.017) | 97.30 (±0.003) |
| 1b | 3901.2 (±0.007) | 98.78 (±0.001) |
| IT-5 | 2262.8 (±0.061) | 97.90 (±0.003) |
| IT-6 | 2768.6 (±0.126) | 98.28 (±0.004) |
| IT-7 | 1293.6 (±0.088) | 96.33 (±0.003) |
| IT-8 | 888.8 (±0.007) | 94.64 (±0.001) |

Table 11. Electrochemical impedance spectroscopy data for compounds 1a, 1b, and IT-5-IT-8.

TABLE 12
Corrosion current data for compounds 1a, 1b, and IT-5-IT-8.

| Inhibitor | E_{corr} (mV) | I_{corr} (μA cm⁻²) | % η_{I} |
|---|---|---|---|
| Blank | −478.0 | 486.27 | — |
| 1a | −442.9 | 8.38 (±0.045) | 98.27 (±0.0009) |
| 1b | −439.2 | 6.62 (±0.094) | 98.64 (±0.0010) |
| IT-5 | −443.2 | 7.94 (±0.118) | 98.37 (±0.0019) |
| IT-6 | −430.9 | 7.93 (±0.226) | 98.37 (±0.0022) |
| IT-7 | −440.1 | 18.18 (±0.026) | 96.26 (±0.0014) |
| IT-8 | −459.6 | 25.86 (±0.037) | 94.68 (±0.0021) |

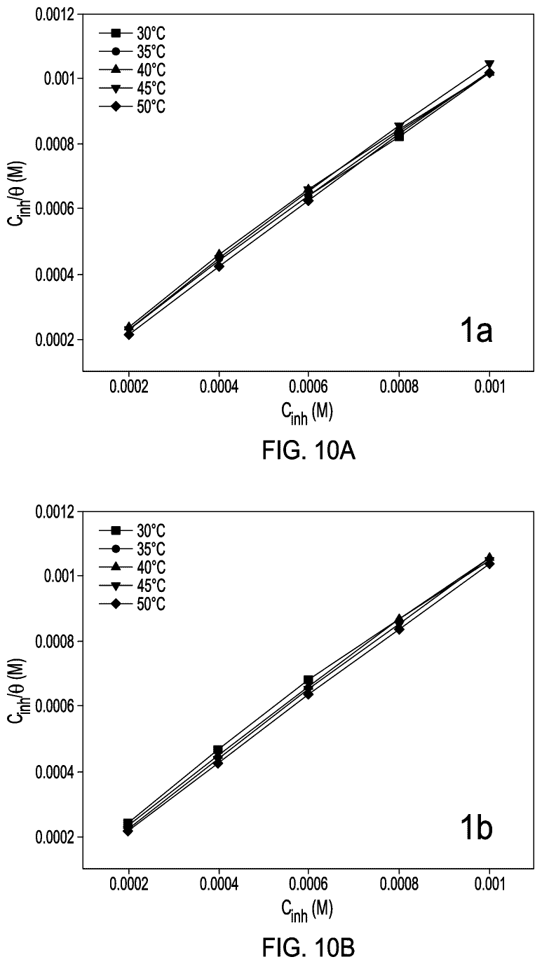

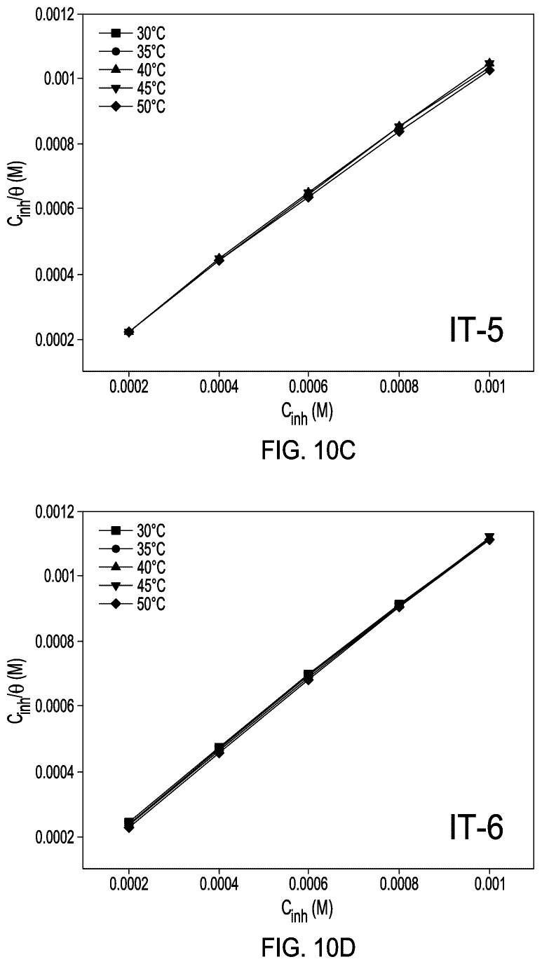

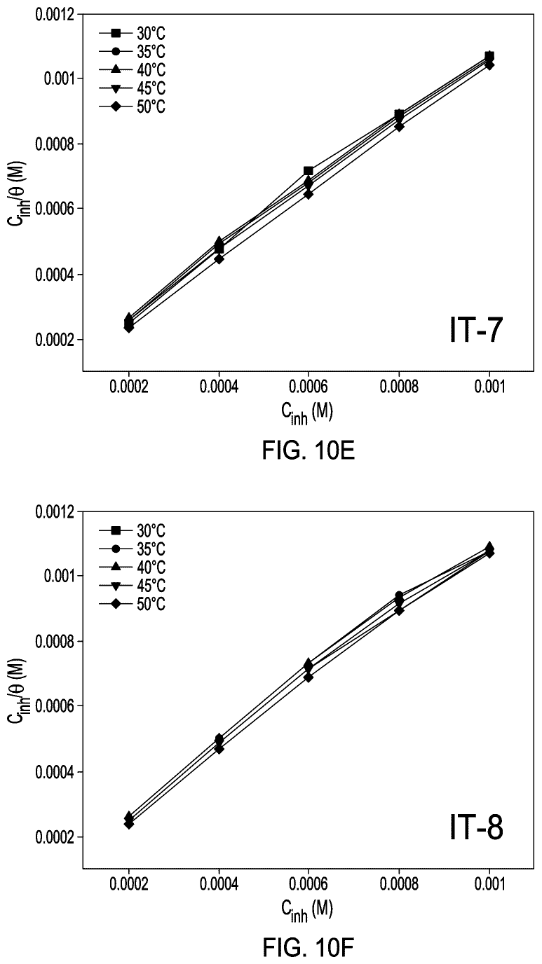

FIGS. 10A-F illustrate Langmuir isotherms for compounds 1a, 1b, and IT-5-IT-8.

TABLE 13
Gibbs free energy at 50° C. for compounds 1a, 1b, and IT-5-IT-8.

| Inhibitor | K_{ads} × 10⁴ (M⁻¹) | ΔG_{ads} (kJ/mol) |
|---|---|---|
| 1a | 5.76 (±0.047) | −39.57 (±0.007) |
| 1b | 6.47 (±0.018) | −40.55 (±0.001) |
| IT-5 | 3.19 (±0.025) | −39.72 (±0.001) |
| IT-6 | 4.96 (±0.013) | −39.92 (+0.001) |
| IT-7 | 2.47 (±0.018) | −37.96 (±0.003) |
| IT-8 | 2.07 (±0.014) | −37.49 (±0.001) |

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### EXEMPLARY ASPECTS

The following exemplary aspects are provided, the numbering of which is not to be construed as designating levels of importance:
Aspect 1 provides a compound for corrosion inhibition of a metal, wherein the compound is an indole-3-thiosemicarbazone.
Aspect 2 provides the corrosion inhibition compound of Aspect 1, wherein the compound is a thiosemicarbazone of an indole-3-carboxaldehyde, wherein the thiosemicarbazone and the indole-3-carboxaldehyde are independently substituted or unsubstituted.
Aspect 3 provides the corrosion inhibition compound of any one of Aspects 1-2, having the structure:

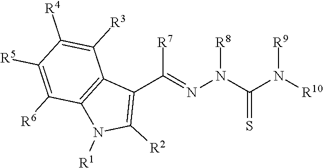

wherein
R¹-R³ and R⁵-R⁸ are independently chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, thiazol-2-yl, pyrimidin-2-yl, benzo[d]thiazol-2-yl, —Cl, —Br, —F, and —I,
R⁴ is chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, thiazol-2-yl, pyrimidin-2-yl, benzo[d]thiazol-2-yl, —Cl, —Br, —F, —I, and —C═N—NH—C(═S)—NH₂,
R⁹ and R¹⁰ are independently chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, 2-thiazol-2-yl, pyrimidin-2-yl, benzo[d]thiazol-2-yl, —Cl, —Br, —F, and —I, or wherein R⁹ and R¹⁰ together with the nitrogen atom to which they are attached form a morpholine ring or a thiomorpholine ring, and
at each occurrence R¹¹ is independently chosen from —O—, —S—, and —NH—.
Aspect 4 provides the corrosion inhibition compound Aspect 3, wherein R¹-R¹⁰ are independently chosen from —H, —OH, —Br, (C₁-C₂₀)alkyl, —O—(C₁-C₂₀)alkyl, (C₆-C₂₀)aryl, —O—(C₆-C₂₀)aryl, (C₁-C₂₀)heteroaryl, and —O—(C₁-C₂₀)heteroaryl.
Aspect 5 provides the corrosion inhibition compound of any one of Aspects 3-4, wherein R¹-R¹⁰ are independently chosen from —H, —OH, Br, —O—(C₁-C₅)alkyl, and (C₁-C₂₀)heteroaryl.
Aspect 6 provides the corrosion inhibition compound of any one of Aspects 3-5, wherein R², R³, R⁵, R⁶, R⁷, R⁸, R⁹, and R¹⁰ are —H.
Aspect 7 provides the corrosion inhibition compound of any one of Aspects 3-6, wherein R⁴ is chosen from —H, —Br, and —OCH₃.
Aspect 8 provides the corrosion inhibition compound of any one of Aspects 3-7, wherein R¹ is chosen from —H, pyrimidin-2-yl, and benzo[d]thiazol-2-yl.
Aspect 9 provides the corrosion inhibition compound of any one of Aspects 3-8, wherein
R²-R³ and R⁶-R¹⁰ are —H,
R⁴ and R⁵ are independently chosen from H, —OCH₃, and —Br,
R⁹ and R¹⁰ are —H, or R⁹ and R¹⁰ together form a morpholine ring, and
R¹ is chosen from —H and (C₁-C₂₀)heteroaryl, such as from —H, pyrimidin-2-yl, and benzol[d]thiazol-2-yl.
Aspect 10 provides the corrosion inhibition compound of any one of Aspects 3-9, wherein
R², R³, R⁵, R⁶, R⁷, R⁸, R⁹, and R¹⁰ are —H,
R¹ is chosen from —H and (C₁-C₂₀)heteroaryl, such as from —H, pyrimidin-2-yl, and benzo[d]thiazol-2-yl, and
R⁴ is chosen from —H and —O—(C₁-C₅)alkyl, such as from —H and —OCH₃.
Aspect 11 provides the corrosion inhibition compound of any one of Aspects 1-10, having the structure:

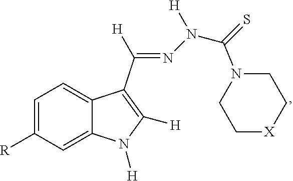

wherein R is —H and X is O.
Aspect 12 provides the corrosion inhibition compound of any one of Aspects 1-10, having the structure:

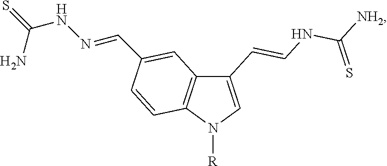

wherein R is —H.
Aspect 13 provides the corrosion inhibition compound of any one of Aspects 1-10, having the structure:

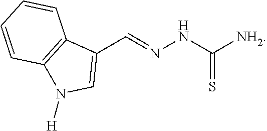 [SMILES: NC(=S)N/N=C/c1c[nH]c2ccccc12]

Aspect 14 provides the corrosion inhibition compound of any one of Aspects 1-10, having the structure:

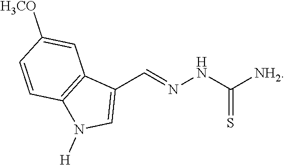 [SMILES: COc1ccc2[nH]cc(/C=N/NC(N)=S)c2c1]

Aspect 15 provides the corrosion inhibition compound of any one of Aspects 1-10, having the structure:

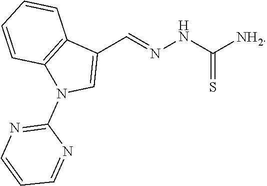 [SMILES: NC(=S)N/N=C/c1cn(-c2ncccn2)c2ccccc12]

Aspect 16 provides the corrosion inhibition compound of any one of Aspects 1-10, having the structure:

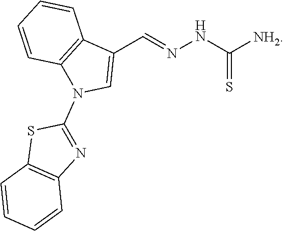 [SMILES: NC(=S)N/N=C/c1cn(-c2nc3ccccc3s2)c2ccccc12]

Aspect 17 provides the corrosion inhibition compound of any one of Aspects 1-10, wherein:
R¹, R³, and R⁶-R¹⁰ are —H,
R² is —H,
R⁴ is —H, and
R⁵ is —OCH₃.
Aspect 18 provides the corrosion inhibition compound of any one of Aspects 1-10, wherein:
R¹, R³, and R⁶-R¹⁰ are —H,
R² is —H,
R⁴ is —Br, and
R⁵ is —H.
Aspect 19 provides the corrosion inhibition compound of any one of Aspects 1-10, wherein:
R¹, R³, and R⁶-R¹⁰ are —H,
R² is —H,
R⁴ is —OH, and
R⁵ is —H.
Aspect 20 provides the corrosion inhibition compound of any one of Aspects 1-10, wherein:
R¹, R³, and R⁶-R¹⁰ are —H,
R² is —CH₃,
R⁴ is —H, and
R⁵ is —H.
Aspect 21 provides a compound for corrosion inhibition of a metal having the following structure:

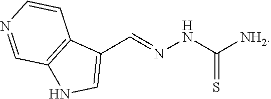 [SMILES: NC(=S)N/N=C/c1c[nH]c2cnccc12]

Aspect 22 provides a compound for corrosion inhibition of a metal having the following structure:

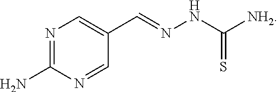 [SMILES: NC(=S)N/N=C/c1cnc(N)nc1]

Aspect 23 provides the corrosion inhibition compound of any one of Aspects 1-22, wherein the compound when contacted with the metal in an aqueous solution comprising a concentration of 1 mM of the compound and comprising 0.1 M H₂SO₄ has an inhibition efficiency (% η_{R}) of 60 to 100.
Aspect 24 provides the corrosion inhibition compound of any one of Aspects 1-22, wherein the compound when contacted with the metal in an aqueous solution comprising a concentration of 1 mM of the compound and comprising 0.1 M H₂SO₄ has an inhibition efficiency (% η_{R}) of 95 to 99.9.
Aspect 25 provides the corrosion inhibition compound of any one of Aspects 1-22, wherein the compound when contacted with the metal in an aqueous solution comprising a concentration of 1 mM of the compound and comprising 0.1 M H₂SO₄ has an inhibition efficiency (% η_{R}) of 97.5 to 99.5.
Aspect 26 provides the corrosion inhibition compound of any one of Aspects 1-25, wherein the compound is a corrosion inhibitor of the metal.
Aspect 27 provides the corrosion inhibition compound of any one of Aspects 1-26, wherein a reaction product of the compound and (a) an acid, or (b) the metal, or (c) a combination thereof, is a corrosion inhibitor of the metal.
Aspect 28 provides the corrosion inhibition compound of any one of Aspects 1-27, wherein a reaction product of the compound and the metal in the presence of an acid is a corrosion inhibitor of the metal.
Aspect 29 provides the corrosion inhibition compound of any one of Aspects 1-28, wherein the metal contacted with the compound under acidic conditions exhibits inhibited corrosion.
Aspect 30 provides the corrosion inhibition compound of Aspect 29, wherein the metal removed from the acidic conditions continues to exhibit inhibited corrosion.
Aspect 31 provides the corrosion inhibition compound of any one of Aspects 1-30, wherein the metal comprises aluminum, copper, iron, zinc, manganese, nickel, chromium, molybdenum, boron, titanium, vanadium, tungsten, cobalt, niobium, an alloy thereof, or a combination thereof.
Aspect 32 provides the corrosion inhibition compound of any one of Aspects 1-31, wherein the metal comprises aluminum, copper, iron, zinc, an alloy thereof, or a combination thereof.
Aspect 33 provides the corrosion inhibition compound of any one of Aspects 1-32, wherein the metal is a steel.
Aspect 34 provides the corrosion inhibition compound of any one of Aspects 1-33, wherein the metal is a carbon steel, an alloy steel, or a combination thereof.
Aspect 35 provides the corrosion inhibition compound of any one of Aspects 1-34, wherein the metal comprises iron and also comprises manganese, nickel, chromium, molybdenum, boron, titanium, vanadium, tungsten, cobalt, niobium, or a combination thereof.
Aspect 36 provides a corrosion inhibition composition comprising the corrosion inhibition compound of any one of Aspects 1-35, wherein the corrosion inhibition composition inhibits corrosion of the metal contacted therewith.
Aspect 37 provides the composition of Aspect 36, wherein the composition is an aqueous composition comprising acid.
Aspect 38 provides the composition of any one of Aspects 36-37, wherein the composition provides inhibited corrosion of the metal during contacting of the composition therewith.
Aspect 39 provides the composition of Aspect 38, wherein the composition provides inhibited corrosion of the metal after removing the metal therefrom.
Aspect 40 provides the composition of any one of Aspects 36-39, wherein the composition is a paint, a coating, a spray, a lubricant composition, or a combination thereof.
Aspect 41 provides a method of inhibiting corrosion, the method comprising:
contacting the metal with the compound of any one of Aspects 1-35 and/or a reaction product thereof.
Aspect 42 provides the method of Aspect 41, wherein the contacting comprises contacting a composition that comprises the compound and/or the reaction product thereof with the metal.
Aspect 43 provides the method of any one of Aspects 41-42, comprising performing the contacting of the metal under acidic conditions.
Aspect 44 provides the method of Aspect 43, wherein the contacting comprises exposing the metal to an acidic solution comprising the corrosion inhibition compound of any one of Aspects 1-35 and/or a reaction product thereof.
Aspect 45 provides the method of Aspect 44, further comprising removing the metal from the acidic solution.
Aspect 46 provides the method of Aspect 45, further comprising rinsing residual acidic solution from the metal after removing the metal from the acidic solution.
Aspect 47 provides the method of any one of Aspects 43-46, wherein the method provides corrosion inhibition of the metal during the contacting of the metal with acidic solution.
Aspect 48 provides the method of any one of Aspects 45-47, wherein the method provides corrosion inhibition of the metal after removal of the metal from the acidic solution.
Aspect 49 provides the method of any one of Aspects 41-48, wherein the metal comprises aluminum, copper, iron, zinc, manganese, nickel, chromium, molybdenum, boron, titanium, vanadium, tungsten, cobalt, niobium, an alloy thereof, or a combination thereof.
Aspect 50 provides the method of any one of Aspects 41-49, wherein the metal comprises aluminum, copper, iron, zinc, an alloy thereof, or a combination thereof.
Aspect 51 provides the method of any one of Aspects 41-50, wherein the metal is a steel.
Aspect 52 provides the method of any one of Aspects 41-51, wherein the metal is a carbon steel, an alloy steel, or a combination thereof.
Aspect 53 provides the method of any one of Aspects 41-52, wherein the metal comprises iron and also comprises manganese, nickel, chromium, molybdenum, boron, titanium, vanadium, tungsten, cobalt, niobium, or a combination thereof.
Aspect 54 provides a metal having a decreased rate of corrosion, the metal comprising:
a coating and/or absorbed layer on an exterior surface of the metal comprising the compound of any one of Aspects 1-35 or a reaction product thereof.
Aspect 55 provides a metal having a decreased rate of corrosion, the metal comprising:
a coating and/or absorbed layer on an exterior surface of the metal comprising a reaction product of the corrosion inhibition compound of any one of Aspects 1-35 and
an acidic solution, or
the exterior surface of the metal, or
a combination thereof.
Aspect 56 provides a method of forming the compound of any one of Aspects 1-20, the method comprising:
reacting a substituted or unsubstituted indole-3-carboxaldehyde with a substituted or unsubstituted thiosemicarbazide to form the compound of any one of Aspects 1-20.
Aspect 57 provides the method of Aspect 56, wherein the indole-3-carboxaldehyde is naturally derived.
Aspect 58 provides the compound or method of any one or any combination of Aspects 1-57 optionally configured such that all elements or options recited are available to use or select from.

What is claimed is:

1. A compound for corrosion inhibition of a metal, wherein the compound is an indole-3-thiosemicarbazone, or wherein the compound has the structure:

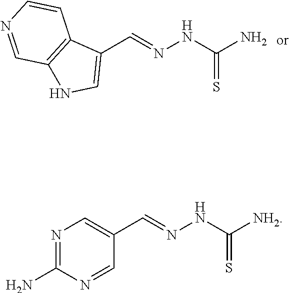 [SMILES: NC(=S)N/N=C/c1c[nH]c2cnccc12 ; NC(=S)N/N=C/c1cnc(N)nc1]

2. The corrosion inhibition compound of claim 1, having the structure: 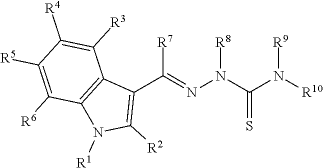
wherein
R¹-R³ and R⁵-R⁸ are independently chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, thiazol-2-yl, pyrimidin-2-yl, benzo[d]thiazol-2-yl, —Cl, —Br, —F, and —I,
R⁴ is chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, thiazol-2-yl, pyrimidin-2-yl, benzo[d]thiazol-2-yl, —Cl, —Br, —F, —I, and —C═N—NH—C(═S)—NH₂,
R⁹ and R¹⁰ are independently chosen from —H, —OH, (C₁-C₂₀)hydrocarbyl, —R¹¹—(C₁-C₂₀)hydrocarbyl, (C₆-C₂₀)aryl, —R¹¹—(C₆-C₂₀)aryl, (C₁-C₂₀)heteroaryl, —R¹¹—(C₁-C₂₀)heteroaryl, 2-thiazol-2-yl, pyrimidin-2-yl, benzo[d]thiazol-2-yl, —Cl, —Br, —F, and —I, or wherein R⁹ and R¹⁰ together with the nitrogen atom to which they are attached form a morpholine ring or a thiomorpholine ring, and
at each occurrence R¹¹ is independently chosen from —O—, —S—, and —NH—.

3. The corrosion inhibition compound of claim 2, wherein
R²-R³ and R⁶-R¹⁰ are —H,
R⁴ and R⁵ are independently chosen from H, —OCH₃, and —Br,
R⁹ and R¹⁰ are —H, or R⁹ and R¹⁰ together form a morpholine ring, and
R¹ is chosen from —H and (C₁-C₂₀)heteroaryl, such as from —H, pyrimidin-2-yl, and benzol[d]thiazol-2-yl.

4. The corrosion inhibition compound of claim 2, wherein
R², R³, R⁵, R⁶, R⁷, R⁸, R⁹, and R¹⁰ are —H,
R¹ is chosen from —H and (C₁-C₂₀)heteroaryl, and
R⁴ is chosen from —H and —O—(C₁-C₅)alkyl.

5. The corrosion inhibition compound of claim 2, wherein:
R¹, R³, and R⁶-R¹⁰ are —H, R² is —H, R⁴ is —H, and R⁵ is —OCH₃; or
R¹, R³, and R⁶-R¹⁰ are —H, R² is —H, R⁴ is —Br, and R⁵ is —H; or
R¹, R³, and R⁶-R¹⁰ are —H, R² is —H, R⁴ is —OH, and R⁵ is —H; or
R¹, R³, and R⁶-R¹⁰ are —H, R² is —CH₃, R⁴ is —H, and R⁵ is —H.

6. The corrosion inhibition compound of claim 1, having the structure:

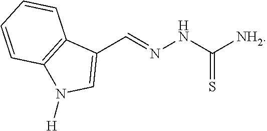 [SMILES: NC(=S)N/N=C/c1c[nH]c2ccccc12]

7. The corrosion inhibition compound of claim 1, having the structure:

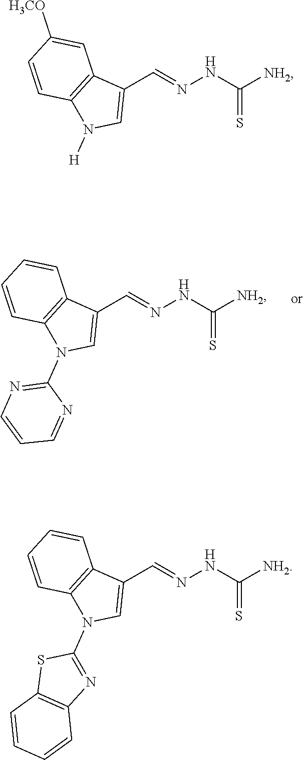 [SMILES: COc1ccc2[nH]cc(/C=N/NC(N)=S)c2c1 ; NC(=S)N/N=C/c1cn(-c2ncccn2)c2ccccc12 ; NC(=S)N/N=C/c1cn(-c2nc3ccccc3s2)c2ccccc12]

8. The corrosion inhibition compound of claim 1, wherein the corrosion inhibition compound has the structure: 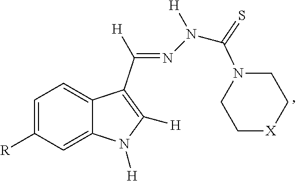
wherein R is —H and X is O, or 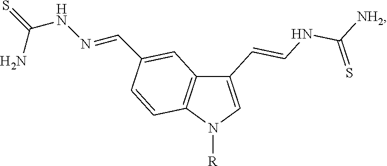
wherein R is —H.

9. The corrosion inhibition compound of claim 1, wherein the corrosion inhibition compound has the structure:

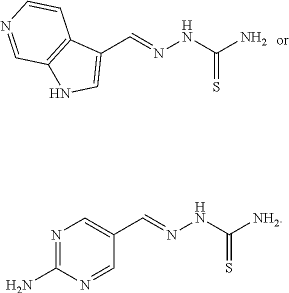 [SMILES: NC(=S)N/N=C/c1c[nH]c2cnccc12 ; NC(=S)N/N=C/c1cnc(N)nc1]

10. The corrosion inhibition compound of claim 1, wherein the compound when contacted with the metal in an aqueous solution comprising a concentration of 1 mM of the compound and comprising 0.1 M H₂SO₄ has an inhibition efficiency (% η_{R}) of 95 to 99.9.

11. The corrosion inhibition compound of claim 1, wherein the compound is a corrosion inhibitor of the metal.

12. The corrosion inhibition compound of claim 1, wherein a reaction product of the compound and (a) an acid, or (b) the metal, or (c) a combination thereof, is a corrosion inhibitor of the metal.

13. A corrosion inhibition composition comprising the corrosion inhibition compound of claim 1, wherein the corrosion inhibition composition inhibits corrosion of the metal contacted therewith.

14. The composition of claim 13, wherein the composition is an aqueous composition comprising acid.

15. A method of inhibiting corrosion, the method comprising:
contacting the metal with the compound of claim 1 and/or a reaction product thereof.

16. The method of claim 15, comprising performing the contacting of the metal under acidic conditions.

17. The method of claim 15, wherein the metal comprises aluminum, copper, iron, zinc, manganese, nickel, chromium, molybdenum, boron, titanium, vanadium, tungsten, cobalt, niobium, an alloy thereof, or a combination thereof.

18. The method of claim 15, wherein the metal is a steel.

19. A metal having a decreased rate of corrosion, the metal comprising:
a coating and/or absorbed layer on an exterior surface of the metal comprising the corrosion inhibition compound of claim 1 or a reaction product thereof.

20. A method of forming the indole-3-thiosemicarbazone of claim 1, the method comprising:
reacting a substituted or unsubstituted indole-3-carboxaldehyde with a substituted or unsubstituted thiosemicarbazide to form the indole-3-thiosemicarbazone of claim 1.
